(19) United States
(12) Patent Application Publication
(10) Pub. No.: US 2010/0104534 A1
(43) Pub. Date: Apr. 29, 2010

(54) HYDRAZIDO-PEPTIDES AS INHIBITORS OF HCV NS3-PROTEASE

(76) Inventors: Srikanth Venkatraman, Edison, NJ (US); F. George Njoroge, Warren, NJ (US); Neng-Yang Shih, Lexington, MA (US)

Correspondence Address: MERCK;PATENT DEPARTMENT (K-6-1, 1990), 2000 GALLOPING HILL ROAD, KENILWORTH, NJ, 07033-0530, US

(21) Appl. No.: 12/532,223

(22) PCT Filed: Mar. 20, 2008
(86) PCT No.: PCT/US08/03654

§ 371 Date: Dec. 1, 2009

Related U.S. Application Data
(60) Provisional application No. 60/919,732, filed on Mar. 23, 2007.

(51) Int. Cl. A61K 38/21; C07D 207/09; A61K 31/40; A61K 31/445; A61K 31/439; C07D 401/12

(52) U.S. Cl. 424/85.7; 548/537; 514/423; 424/85.4; 548/515; 514/412; 514/323; 514/299; 546/208; 546/112

(57) ABSTRACT

The present invention discloses novel compounds, which have HCV protease inhibitory activity as well as methods for preparing such compounds. In another embodiment, the invention discloses pharmaceutical compositions comprising such compounds as well as methods of using them to treat disorders associated with the HCV protease.

### FIELD OF THE INVENTION

The present invention relates to novel hepatitis C virus (“HCV”) protease inhibitors, pharmaceutical compositions containing one or more such inhibitors, methods of preparing such inhibitors and methods of using such inhibitors to treat hepatitis C and related disorders. This invention additionally discloses novel macrocyclic compounds as inhibitors of the HCV NS3/NS4a serine protease. This application claims priority from U.S. provisional patent application Ser. No. 60/919,732, filed Mar. 23, 2007.

### BACKGROUND OF THE INVENTION

Hepatitis C virus (HCV) is a (+)-sense single-stranded RNA virus that has been implicated as the major causative agent in non-A, non-B hepatitis (NANBH), particularly in blood-associated NANBH (BB-NANBH) (see, International Patent Application Publication No. WO 89/04669 and European Patent Application Publication No. EP 381 216). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

Recently, an HCV protease necessary for polypeptide processing and viral replication has been identified, cloned and expressed. (See, e.g., U.S. Pat. No. 5,712,145). This approximately 3000 amino acid polyprotein contains, from the amino terminus to the carboxy terminus, a nucleocapsid protein (C), envelope proteins (E1 and E2) and several non-structural proteins (NS1, 2, 3, 4a, 5a and 5b). NS3 is an approximately 68 kda protein, encoded by approximately 1893 nucleotides of the HCV genome, and has two distinct domains: (a) a serine protease domain consisting of approximately 200 of the N-terminal amino acids; and (b) an RNA-dependent ATPase domain at the C-terminus of the protein. The NS3 protease is considered a member of the chymotrypsin family because of similarities in protein sequence, overall three-dimensional structure and mechanism of catalysis. Other chymotrypsin-like enzymes are elastase, factor Xa, thrombin, trypsin, plasmin, urokinase, tPA and PSA. The HCV NS3 serine protease is responsible for proteolysis of the polypeptide (polyprotein) at the NS3/NS4a, NS4a/NS4b, NS4b/NS5a and NS5a/NS5b junctions and is thus responsible for generating four viral proteins during viral replication. This has made the HCV NS3 serine protease an attractive target for antiviral chemotherapy. The inventive compounds can inhibit such protease. They also can modulate the processing of hepatitis C virus (HCV) polypeptide.

It has been determined that the NS4a protein, an approximately 6 kda polypeptide, is a co-factor for the serine protease activity of NS3. Autocleavage of the NS3/NS4a junction by the NS3/NS4a serine protease occurs intramolecularly (i.e., cis) while the other cleavage sites are processed intermolecularly (i.e., trans).

Analysis of the natural cleavage sites for HCV protease revealed the presence of cysteine at P1 and serine at P1′ and that these residues are strictly conserved in the NS4a/NS4b, NS4b/NS5a and NS5a/NS5b junctions. The NS3/NS4a junction contains a threonine at P1 and a serine at P1′. The Cys→Thr substitution at NS3/NS4a is postulated to account for the requirement of cis rather than trans processing at this junction. See, e.g., Pizzi et al. (1994) Proc. Natl. Acad. Sci. (USA) 91:888-892, Fulla et al. (1996) Folding & Design 1:35-42. The NS3/NS4a cleavage site is also more tolerant of mutagenesis than the other sites. See, e.g., Kollykhalov et al. (1994) J. Virol. 68:7525-7533. It has also been found that acidic residues in the region upstream of the cleavage site are required for efficient cleavage. See, e.g., Komoda et al. (1994) J. Virol. 68:7351-7357.

Inhibitors of HCV protease that have been reported include antioxidants (see, International Patent Application Publication No. WO 98/14181), certain peptides and peptide analogs (see, International Patent Application Publication No. WO 98/17679, Landro et al. (1997) Biochem. 36:9340-9348, Ingallinella et al. (1998) Biochem. 37:8906-8914, Llinàs-Brunet et al. (1998) Bioorg. Med. Chem. Lett. 8:1713-1718), inhibitors based on the 70-amino acid polypeptide eglin c (Martin et al. (1998) Biochem. 37:11459-11468, inhibitors affinity selected from human pancreatic secretory trypsin inhibitor (hPSTI-C3) and minibody repertoires (MBip) (Dimasi et al. (1997) J. Virol. 71:7461-7469), cV_{H}E2 (a “camelized” variable domain antibody fragment) (Martin et al. (1997) Protein Eng. 10:607-614), and α1-antichymotrypsin (ACT) (Elzouki et al.) (1997) J. Hepat. 27:42-28). A ribozyme designed to selectively destroy hepatitis C virus RNA has recently been disclosed (see, BioWorld Today 9(217): 4 (Nov. 10, 1998)).

Reference is also made to the PCT Publications, No. WO 98/17679, published Apr. 30, 1998 (Vertex Pharmaceuticals Incorporated); WO 98/22496, published May 28, 1998 (F. Hoffmann-La Roche AG); and WO 99/07734, published Feb. 18, 1999 (Boehringer Ingelheim Canada Ltd.).

HCV has been implicated in cirrhosis of the liver and in induction of hepatocellular carcinoma. The prognosis for patients suffering from HCV infection is currently poor. HCV infection is more difficult to treat than other forms of hepatitis due to the lack of immunity or remission associated with HCV infection. Current data indicates a less than 50% survival rate at four years post cirrhosis diagnosis. Patients diagnosed with localized resectable hepatocellular carcinoma have a five-year survival rate of 10-30%, whereas those with localized unresectable hepatocellular carcinoma have a five-year survival rate of less than 1%.

Reference is made to WO 00/59929 (U.S. Pat. No. 6,608,027, Assignee: Boehringer Ingelheim (Canada) Ltd.; Published Oct. 12, 2000) which discloses peptide derivatives of the formula:

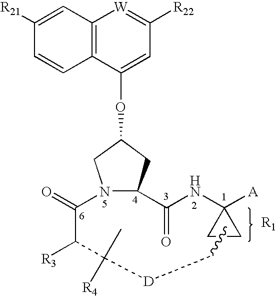

Reference is made to A. Marchetti et al, Synlett, 51, 1000-1002 (1999) describing the synthesis of bicylic analogs of an inhibitor of HCV NS3 protease. A compound disclosed therein has the formula:

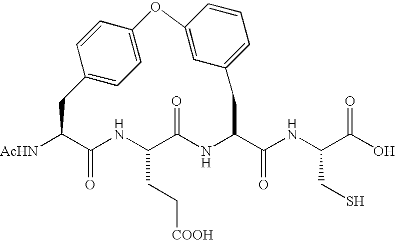

Reference is also made to W. Han et al, Bioorganic & Medicinal Chem. Lett, (2000) 10, 711-713, which describes the preparation of certain α-ketoamides, α-ketoesters and α-diketones containing allyl and ethyl functionalities.

Reference is also made to WO 00/09558 (Assignee: Boehringer Ingelheim Limited; Published Feb. 24, 2000) which discloses peptide derivatives of the formula:

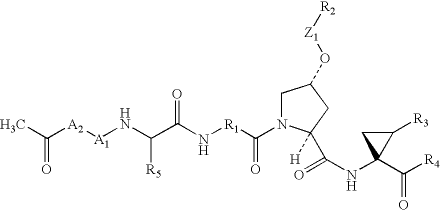

where the various elements are defined therein. An illustrative compound of that series is:

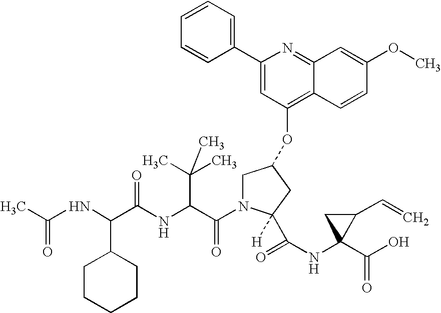

Reference is also made to WO 00/09543 (Assignee: Boehringer Ingelheim Limited; Published Feb. 24, 2000) which discloses peptide derivatives of the formula:

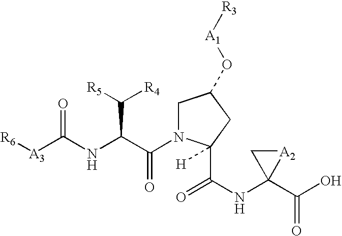

where the various elements are defined therein. An illustrative compound of that series is:

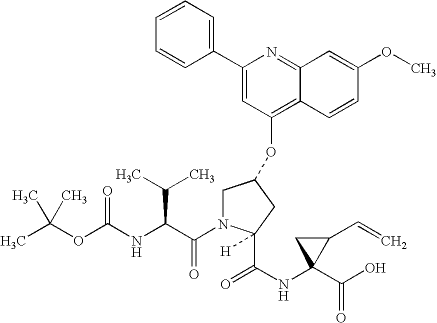

Reference is also made to U.S. Pat. No. 6,608,027 (Boehringer Ingelheim, Canada) which discloses NS3 protease inhibitors of the type:

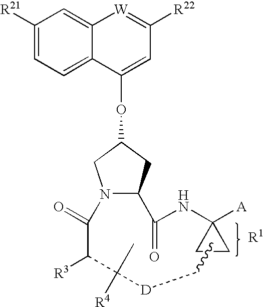

wherein the various moieties are defined therein.

Current therapies for hepatitis C include interferon-α (INF_{α}) and combination therapy with ribavirin and interferon. See, e.g., Beremguer et al. (1998) Proc. Assoc. Am. Physicians 110(2):98-112. These therapies suffer from a low sustained response rate and frequent side effects. See, e.g., Hoofnagle et al. (1997) N. Engl. J. Med. 336:347. Currently, no vaccine is available for HCV infection.

Reference is further made to WO 01/74768 (Assignee: Vertex Pharmaceuticals Inc) published Oct. 11, 2001, which discloses certain compounds of the following general formula (R is defined therein) as NS3-serine protease inhibitors of Hepatitis C virus:

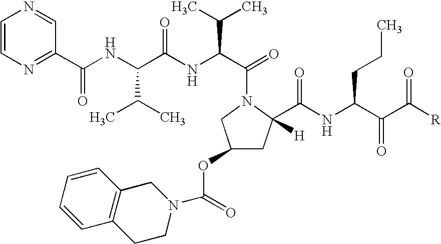

A specific compound disclosed in the afore-mentioned WO 01/74768 has the following formula:

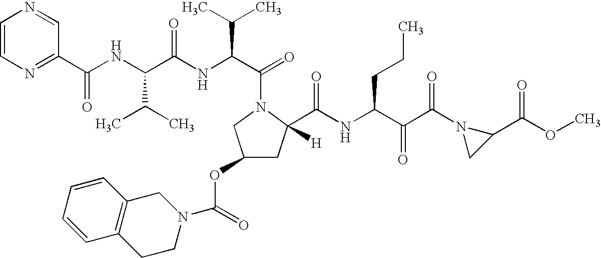

PCT Publications WO 01/77113; WO 01/0 81325; WO 02/08198; WO 02/08256; WO 02/08187; WO 02/08244; WO 02/48172; WO 02/08251; WO 03/062265; WO 05/085275; WO 05/087721; WO 05/087725; WO 05/085242; WO 05/087731; WO 05/058821; WO 05/087730; WO 05/085197; and WO 06/026352, disclose various types of peptides and/or other compounds as NS-3 serine protease inhibitors of hepatitis C virus. The disclosures of those applications are incorporated herein by reference thereto.

There is a need for new treatments and therapies for HCV infection. There is a need for compounds useful in the treatment or prevention or amelioration of one or more symptoms of hepatitis C.

There is a need for methods of treatment or prevention or amelioration of one or more symptoms of hepatitis C.

There is a need for methods for modulating the activity of serine proteases, particularly the HCV NS3/NS4a serine protease, using the compounds provided herein.

There is a need for methods of modulating the processing of the HCV polypeptide using the compounds provided herein.

### SUMMARY OF THE INVENTION

In its many embodiments, the present invention provides a novel class of inhibitors of the HCV protease, pharmaceutical compositions containing one or more of the compounds, methods of preparing pharmaceutical formulations comprising one or more such compounds, and methods of treatment or prevention of HCV or amelioration of one or more of the symptoms of hepatitis C using one or more of such compounds or one or more of such formulations. Also provided are methods of modulating the interaction of an HCV polypeptide with HCV protease. Among the compounds provided herein, compounds that inhibit HCV NS3/NS4a serine protease activity are preferred.

The present invention discloses compounds having the general structure shown in structural Formula I:

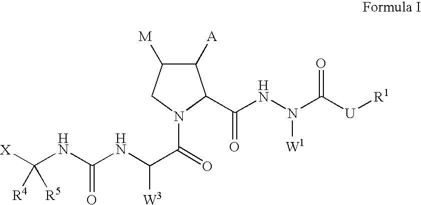

wherein:
R¹ is hydrogen, alkyl, alkenyl, alkynyl, aryl, arylalkyl, arylalkenyl, heteroalkyl, cycloalkenyl, cycloalkenylalkyl-, cycloalkenylalkenyl-, cycloalkyl, cycloalkylalkyl-,
   cycloalkylalkenyl-,

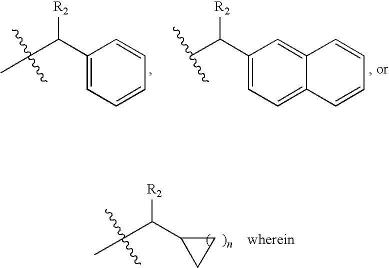

wherein each of said, alkyl, alkenyl, alkynyl, aryl, arylalkyl, arylalkenyl, heteroalkyl, cycloalkenyl, cycloalkenylalkyl-, cycloalkenylalkenyl-, cycloalkyl, cycloalkylalkyl-,
cycloalkylalkenyl-,

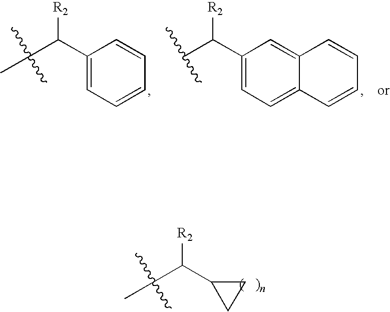

can be unsubstituted or substituted with one or moieties, which can be the same or different, each moiety being independently selected from the group consisting of halogen, nitro, alkyl, aminoalkyl, alkoxyalkyl-, aminoalkloxyalkyl-, alkenyl, heteroaryl, cycloalkyl, cycloalkenyl, heterocycloalkyl, heterocycloalkenyl, aryl, trihaloalkyl, dihaloalkyl, monohaloalkyl, alkylsulfonyl, and arylsulfonyl,
   further wherein R² is selected from the group consisting of hydrogen, alkyl, cycloalkyl, alkenyl, alkynyl, cycloalkenyl, heteroalkyl, heterocycloalkyl, heterocycloalkenyl, aryl, heteroaryl,
   cycloalkylalkyl-, cycloalkenylalkyl-, cycloalkylalkenyl-, cycloalkenylalkenyl-, heterocycloalkylalkyl-, heterocycloalkenylalkyl-, heterocycloalkylalkenyl-, heterocycloalkenylalkenyl-, arylalkyl, heteroaryl, and heteroarylalkyl-;
      A and M are connected to each other such that the moiety:

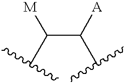

shown above in Formula I, forms either a three, four, five, six, seven or eight-membered cycloalkyl, a three, five, four, six, seven or eight-membered cycloalkenyl, a four to eight-membered heterocyclyl, a four to eight-membered heterocycloalkenyl, a six to ten-membered aryl, or a five to ten-membered heteroaryl wherein each of said three, four, five, six, seven or eight-membered cycloalkyl, three, four, five, six, seven or eight-membered cycloalkenyl, four to eight-membered heterocyclyl, four to eight-membered heterocycloalkenyl, six to ten-membered aryl, or five to ten-membered heteroaryl can be unsubstituted or substituted with one or more moieties, which can be the same or different, each moiety being independently selected from the group consisting of alkyl, alkenyl, alkynyl, halogen, trihaloalkyl, dihaloalkyl, monohaloalkyl, heteroalkyl, amino, aminoalkyl, alkoxyalkyl-, alkylsulfonyl-, and arylsulfonyl-;
   X is selected from the group consisting of:

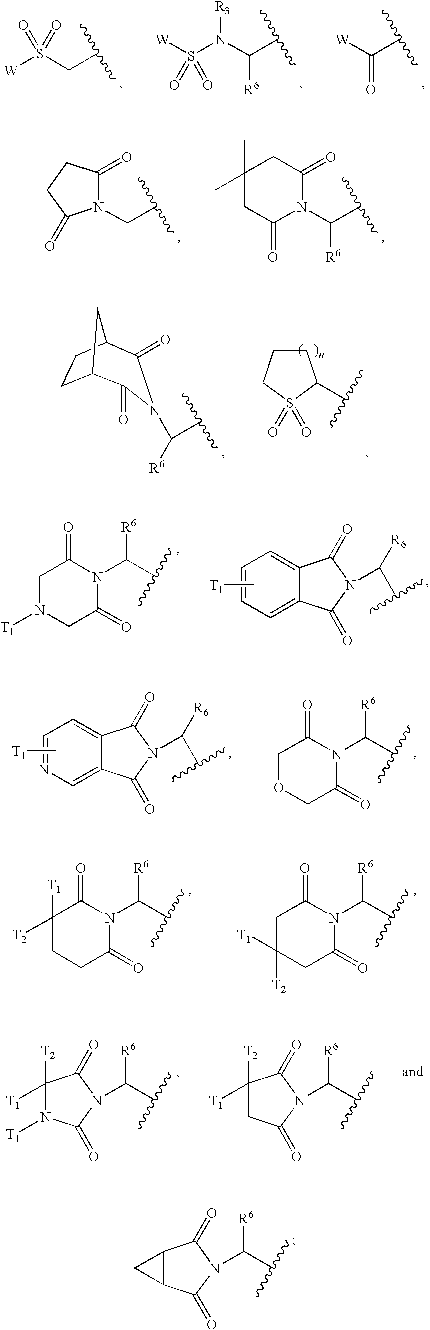

where T₁ and T₂ can be the same or different, each being independently selected from alkyl, aryl, heteroalkyl, heteroaryl, halo, amino, alkylamino-, alkylthio-, amido or carbamate urea;
W³ and R⁶ can be one or two moieties;
W is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
W¹ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
W³ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
R³ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
R⁴ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
R⁵ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
R⁶ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
or R⁴ and R⁵ together with the carbon to which they are attached form either a three to eight-membered cycloalkyl, a four to eight-membered heterocyclyl, three to eight-membered cycloalkenyl, a four to eight-membered heterocycloalkenyl, a six to ten membered aryl, or a five to ten-membered heteroaryl, wherein each of said three to eight-membered cycloalkyl, four to eight-membered heterocyclyl, three to eight-membered cycloalkenyl, four to eight-membered heterocycloalkenyl, six to ten membered aryl, or five to ten-membered heteroaryl can be unsubstituted or substituted with one or more moieties, which can be the same or different, each moiety being independently selected from the group consisting of alkyl, alkenyl, alkynyl, monohaloalkyl, dihaloalkyl, trihaloalkyl and halogen; or

the moiety:

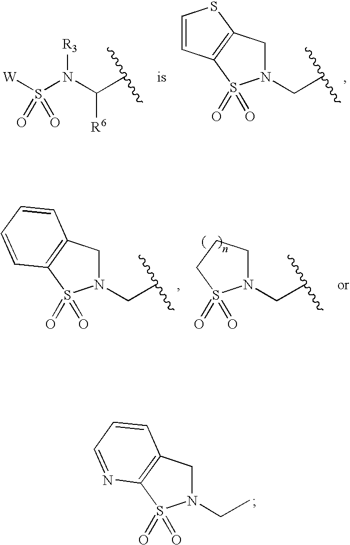

U is selected from the group consisting of O, NR³, S, and CR³₂; and

n is 0-5.

The compounds represented by Formula I, by themselves or in combination with one or more other suitable agents disclosed herein, can be useful for treating diseases such as, for example, HCV, HIV, AIDS (Acquired Immune Deficiency Syndrome), and related disorders, as well as for modulating the activity of hepatitis C virus (HCV) protease, preventing HCV infection, or ameliorating one or more symptoms of hepatitis C. Such modulation, treatment, prevention or amelioration can be done with the inventive compounds as well as with pharmaceutical compositions or formulations comprising such compounds. Without being limited to theory, it is believed that the HCV protease may be the NS3 or NS4a protease. The inventive compounds can inhibit such protease. They can also modulate the processing of hepatitis C virus (HCV) polypeptide.

### DETAILED DESCRIPTION

In an embodiment, the present invention discloses compounds which are represented by structural Formula I or a pharmaceutically acceptable salt, solvate or ester thereof, wherein the various moieties are as defined above.

In another embodiment, in Formula I, R¹ is

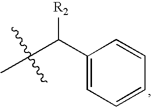

wherein R² is methyl.

In another embodiment, in Formula I, R¹ is

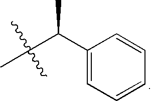

In another embodiment, in Formula I, W¹ is alkyl.

In another embodiment, in Formula I, W¹ is propyl.

In another embodiment, in Formula I, W¹ is cycloalkylalkyl.

In another embodiment, in Formula I, W¹ is cyclopropylmethyl.

In another embodiment, in Formula I, W³ is alkyl.

In another embodiment, in Formula I, W³ is tertiary butyl.

In another embodiment, in Formula I, W³ is cycloalkyl, wherein said cycloalkyl can be unsubstituted or substituted with alkyl.

In another embodiment, in Formula I, W³ is cyclohexyl.

In another embodiment, in Formula I, W³ is

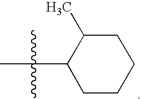

In another embodiment, in Formula I, the moiety:

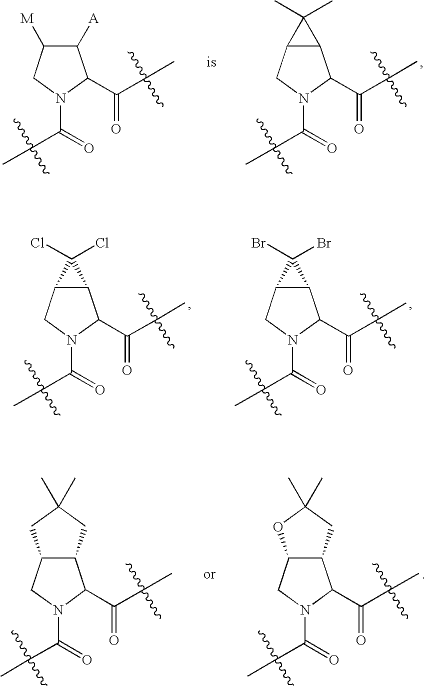

In another embodiment, in Formula I, the moiety:

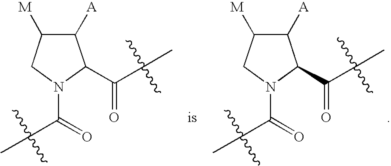

In another embodiment, in Formula I, the moiety:

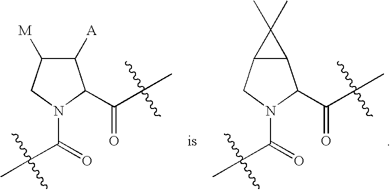

In another embodiment, in Formula I, the moiety:

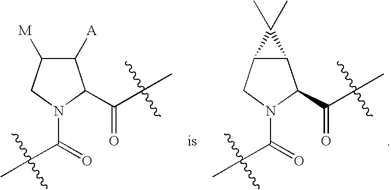

In another embodiment, in Formula I, R⁴ and R⁵ are independently hydrogen or alkyl.

In another embodiment, in Formula I, R⁴ and R⁵ are independently hydrogen or tertiary butyl.

In another embodiment, in Formula I, R⁴ and R⁵ together with the carbon to which they are attached form cycloalkyl.

In another embodiment, in Formula I, R⁴ and R⁵ together with the carbon to which they are attached form cyclohexyl.

In another embodiment, in Formula I, X is selected from the group consisting of

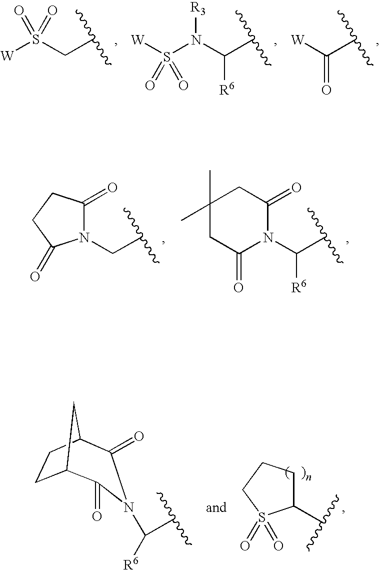

wherein W and R³, which can be the same or different, are independently alkyl or heteroaryl.

In another embodiment, in Formula I, X is

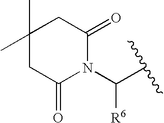

wherein R⁶ are two hydrogens.

In another embodiment, in Formula I, X is

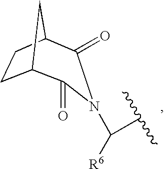

wherein R⁶ are two hydrogens.

In another embodiment, in Formula I, X is

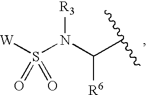

is wherein W and R³ are each independently methyl and R⁶ are two hydrogens.

In another embodiment, in Formula I, X is

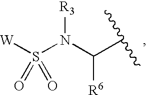

wherein W is pyridyl, R³ is methyl and R⁶ are two hydrogens.

In another embodiment, in Formula I, X is

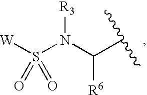

wherein W is

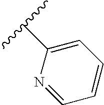

and R³ is methyl and R⁶ are two hydrogens.

In another embodiment, in Formula I, X is

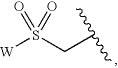

wherein W is tertiary butyl.

In another embodiment, in Formula I, U is NH.

In the embodiments shown below, where moieties for more than one variable is listed for the same embodiment, each variable should be considered as being selected independent of one another.

In another embodiment, this invention discloses a compound of the formula:

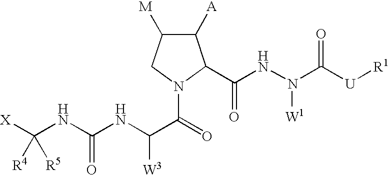

wherein the variable moieties are independently selected, further wherein R¹ is

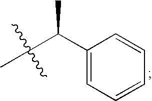

W¹ is propyl or cyclopropylmethyl;

the moiety:

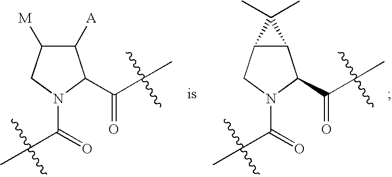

W³ is tertiary butyl, cyclohexyl, or 1-methylcyclohexyl;

R⁴ is hydrogen and R⁵ is tertiary butyl; or R⁴ and R⁵ together with the carbon to which they are attached form cyclohexyl;

X is

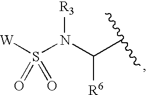

wherein W is methyl or

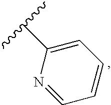

R³ is methyl, and R⁶ are two hydrogen;

and U is NH.

In another embodiment, this invention discloses a compound of the formula:

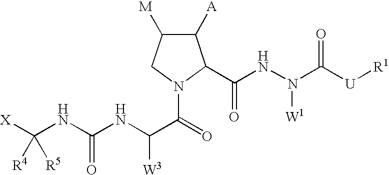

wherein the variable moieties are independently selected, further wherein R¹ is

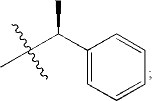

W¹ is propyl;

the moiety:

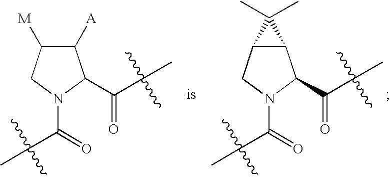

W³ is tertiary butyl;

R⁴ is hydrogen and R⁵ is tertiary butyl;

X is

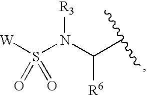

wherein W is methyl, R³ is methyl and R⁶ are two hydrogens;

### and U is NH.

In another embodiment, this invention discloses a compound of the formula:

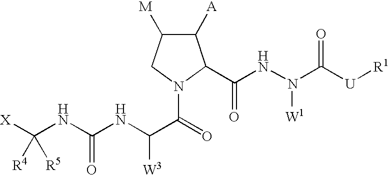

wherein the variable moieties are independently selected, further wherein R¹ is

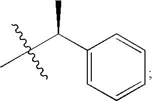

W¹ is propyl;

the moiety:

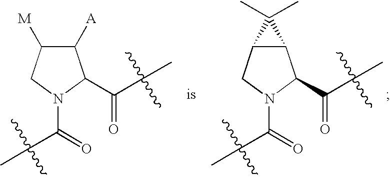

W³ is cyclohexyl;

R⁴ is hydrogen and R⁵ is tertiary butyl;

X is

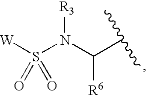

wherein W is

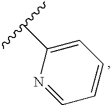

R³ is methyl and R⁶ are two hydrogens;

and U is NH.

In another embodiment, this invention discloses a compound of the formula:

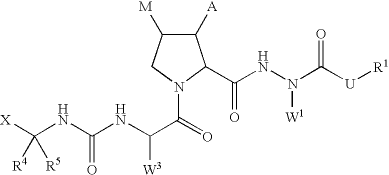

wherein the variable moieties are independently selected, further wherein R¹ is

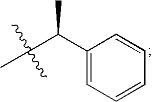

W¹ is propyl or cyclopropylmethyl;

the moiety:

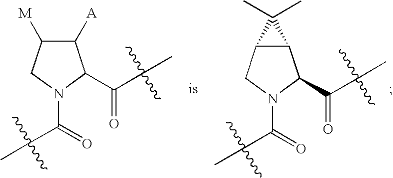

W³ is tertiary butyl, cyclohexyl, or 1-methylcyclohexyl;

R⁴ is hydrogen and R⁵ is tertiary butyl; or R⁴ and R⁵ together with the carbon to which they are attached form cyclohexyl;

X is

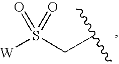

wherein W is tertiary butyl;

and U is NH.

In another embodiment, this invention discloses a compound of the formula:

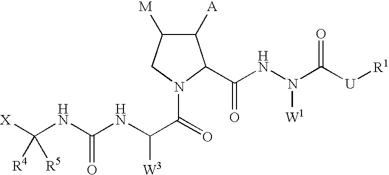

wherein the variable moieties are independently selected, further wherein R¹ is

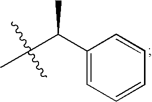

W¹ is propyl or cyclopropylmethyl;

the moiety:

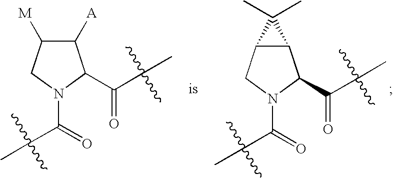

W³ is tertiary butyl, cyclohexyl, or 1-methylcyclohexyl;

R⁴ is hydrogen and R⁵ is tertiary butyl; or R⁴ and R⁵ together with the carbon to which they are attached form cyclohexyl;

X is

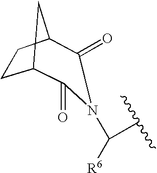

wherein R⁶ are two hydrogens;

and U is NH.

In another embodiment, this invention discloses a compound of the formula:

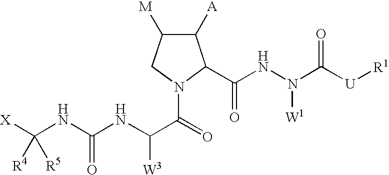

wherein the variable moieties are independently selected, further wherein R¹ is

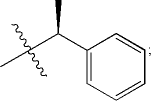

W¹ is propyl;

the moiety:

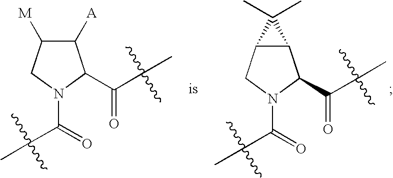

W³ is tertiary butyl, cyclohexyl, or 1-methylcyclohexyl;

R⁴ is hydrogen and R⁵ is independently tertiary butyl; or R⁴ and R⁵ together with the carbon to which they are attached form cyclohexyl;

X is

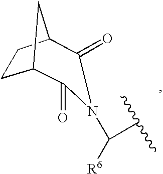

wherein R⁶ are two hydrogens;

and U is NH.

In another embodiment a compound of the formula:

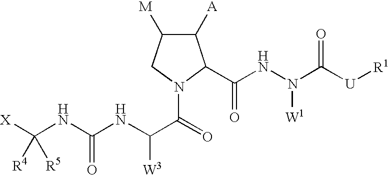

wherein the variable moieties are independently selected, further wherein R¹ is

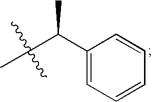

W¹ is cyclopropylmethyl;

the moiety:

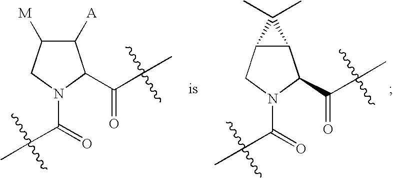

W³ is tertiary butyl, cyclohexyl, or 1-methylcyclohexyl;

R⁴ is hydrogen and R⁵ is tertiary butyl; or R⁴ and R⁵ together with the carbon to which they are attached form cyclohexyl;

X is

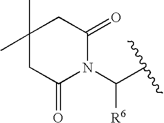

wherein R⁶ are two hydrogens;

and U is NH.

In another embodiment, this invention discloses a compound of the formula:

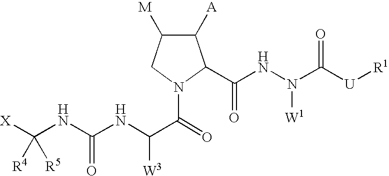

wherein the variable moieties are independently selected, further wherein R¹ is

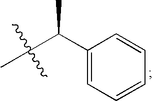

W¹ is propyl or cyclopropylmethyl;

the moiety:

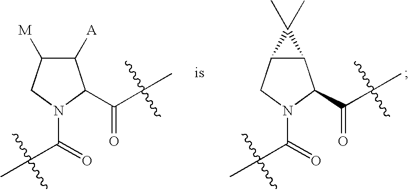

W³ is tertiary butyl;

R⁴ is hydrogen and R⁵ is tertiary butyl;

X is

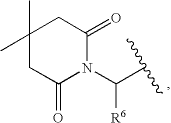

wherein R⁶ are two hydrogens;

and U is NH.

In an additional embodiment, this invention discloses the following compounds in Table 1:

TABLE 1

| Compound | |
|---|---|
| No. | Structure |
| 1 | 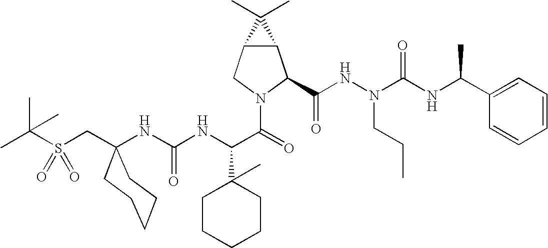 |
| 2 | 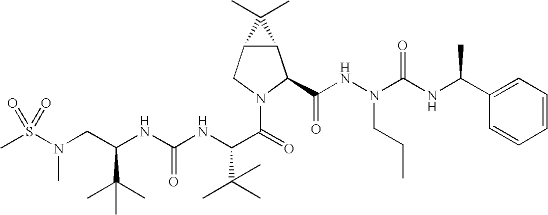 |
| 3 | 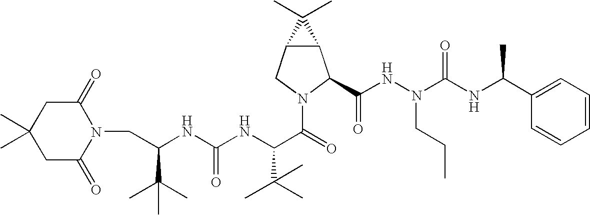 |
| 4 | 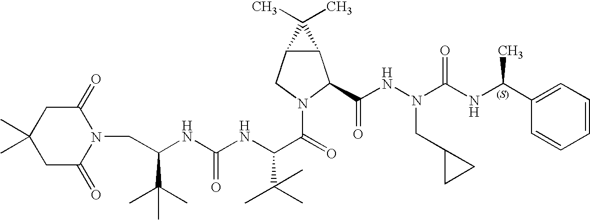 |
| 5 | 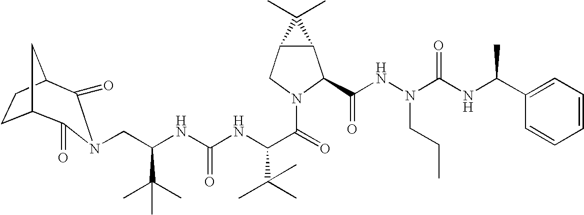 |
| 6 | 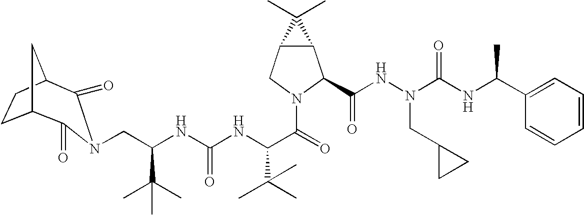 |
| 7 | 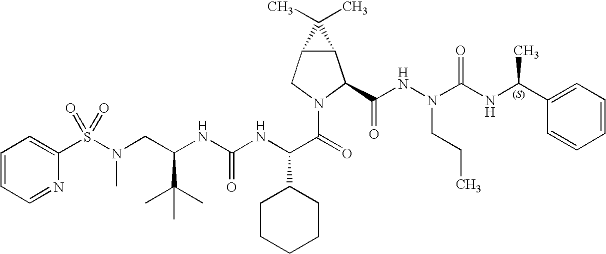 |

Representative compounds according to the invention which exhibit excellent HCV protease inhibitory activity are listed later in this Description in Table 2 along with their biological activity in HCV continuous assay (ranges of Ki* values in nanomolar, nM).

As used above, and throughout this disclosure, the following terms, unless otherwise indicated, shall be understood to have the following meanings:

“Patient” includes both human and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkoxyalkoxy, alkylthio, amino, —NH(alkyl), —NH(cycloalkyl), —N(alkyl)₂, carboxy and C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl, carbazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkylalkenyl” means a cycloalkyl moiety as defined above linked via an alkenyl moiety (defined above) to a parent core.

“Cycloalkenyl” or “cyclenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” or “cyclenylalkyl” means a cycloalkenyl or cyclenyl moiety as defined above linked via an alkyl moiety (defined above) to a parent core. Non-limiting examples of suitable cycloalkenylalkyls include cyclopentenylmethyl, cyclohexenylmethyl and the like.

“Cycloalkenylalkenyl” or “cyclenylalkenyl” means a cycloalkenyl or cyclenyl moiety as defined above linked via an alkenyl moiety (defined above) to a parent core.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, alkoxyalkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

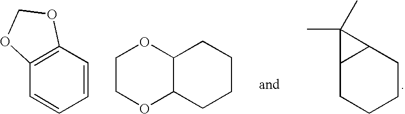

“Heteroalkyl” is a saturated or unsaturated chain containing carbon and at least one heteroatom, wherein one or more of the chain atoms is an element other than carbon, for example nitrogen, oxygen or sulfur, alone or in combination, wherein no two heteroatoms are adjacent. Heteroalkyl chains contain from 2 to 15 member atoms (carbon and heteroatoms) in the chain, preferably 2 to 10, more preferably 2 to 5. For example, alkoxy (i.e., —O-alkyl or —O-heteroalkyl) radicals are included in heteroalkyl. Heteroalkyl chains may be straight or branched. Preferred branched heteroalkyl have one or two branches, preferably one branch. Preferred heteroalkyl are saturated. Unsaturated heteroalkyl have one or more carbon-carbon double bonds and/or one or more carbon-carbon triple bonds. Preferred unsaturated heteroalkyls have one or two double bonds or one triple bond, more preferably one double bond. Heteroalkyl chains may be unsubstituted or substituted with from 1 to 4 substituents. Preferred substituted heteroalkyl are mono-, di-, or tri-substituted. Heteroalkyl may be substituted with lower alkyl, haloalkyl, halo, hydroxy, aryloxy, heteroaryloxy, acyloxy, carboxy, monocyclic aryl, heteroaryl, cycloalkyl, heterocyclyl, spirocycle, amino, acylamino, amido, keto, thioketo, cyano, or any combination thereof

“Heterocyclyl” or “Heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for, example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a single moiety (e.g., carbonyl) which simultaneously replaces two available hydrogens on the same carbon atom on a ring system. Example of such moiety is pyrrolidone:

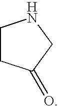

“Heterocyclylalkyl” or “Heterocycloalkylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclylalkenyl” or “Heterocycloalkylalkenyl” means a heterocyclyl moiety as defined above linked via an alkenyl moiety (defined above) to a parent core.

“Heterocyclenyl” or “Heterocycloalkenyl” means a non-aromatic monocyclic or multicyclic ring system comprising about 3 to about 15 ring atoms, preferably about 5 to about 14 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 13 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

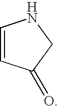

“Heterocyclenylalkyl” means a heterocyclenyl moiety as defined above linked via an alkyl moiety (defined above) to a parent core.

“Heterocyclenylalkenyl” means a heterocyclenyl moiety as defined above linked via an alkenyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

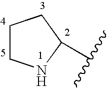

there is no —OH attached directly to carbons marked 2 and 5.

It should also be noted that tautomeric forms such as, for example, the moieties:

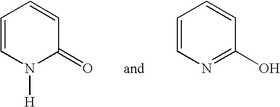

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Spiro ring systems” have two or more rings linked by one common atom. Preferred Spiro ring systems include spiroheteroaryl, spiroheterocyclenyl, spiroheterocyclyl, spirocycloalkyl, spirocyclenyl, and spiroaryl. Non-limiting examples of suitable spiro ring systems include

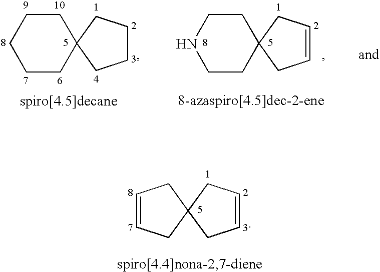

“Amine” is a type of functional group that contains a nitrogen as the key atom. Structurally it resembles ammonia, wherein one or more hydrogen atoms are replaced by organic substituents such as alkyl, cycloalkyl, aryl or any of the other organic substituents defined herein. “Amino” is the amine, as defined above, as a functional group or substituent.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1- naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen. An alkoxy linked directly to another alkoxy is an “alkoxyalkoxy”.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” or “thioalkoxy” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

A carbamate group means a —O—C(O)—N(alkyl or aryl)- group, and a urea group means a —N(alkyl or aryl)-C(O)—N(alkyl or aryl)- group.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound' or “stable structure” is meant a compound that is sufficiently robust to survive isolation to a useful degree of purity from a reaction mixture, and formulation into an efficacious therapeutic agent.

The term “one or more” or “at least one”, when indicating the number of substituents, compounds, combination agents and the like, refers to at least one, and up to the maximum number of chemically and physically permissible, substituents, compounds, combination agents and the like, that are present or added, depending on the context. Such techniques and knowledge are well known within the skills of the concerned artisan.

The term “optionally substituted” means optional substitution with the specified groups, radicals or moieties.

The term “isolated” or “in isolated form” for a compound refers to the physical state of said compound after being isolated from a synthetic process or natural source or combination thereof. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

It should also be noted that any carbon or heteroatom with unsatisfied valences in the text, schemes, examples and Tables herein is assumed to have the hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or compound according to the invention, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds according to the invention are also contemplated herein. The term “prodrug”, as employed herein, denotes a compound that is a drug precursor which, upon administration to a subject, undergoes chemical conversion by metabolic or chemical processes to yield a compound according to the invention or a salt and/or solvate thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” is meant to describe an amount of compound or a composition of the present invention effective in inhibiting the CDK(s) and thus producing the desired therapeutic, ameliorative, inhibitory or preventative effect.

The compounds according to the invention can form salts which are also within the scope of this invention. Reference to a compound according to the invention herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound according to the invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the invention may be formed, for example, by reacting a compound according to the invention with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope according to the invention and all acid and base salts are considered equivalent to the free forms of the corresponding compounds for purposes according to the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds according to the invention, and salts, solvates, esters and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates, esters and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). Individual stereoisomers of the compounds according to the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, positional isomers, racemates or prodrugs of the inventive compounds.

Polymorphic forms of the compounds of Formula I, and of the salts, solvates, esters and prodrugs of the compounds of Formula I, are intended to be included in the present invention.

It is to be understood that the utility of the compounds according to the invention for the therapeutic applications discussed herein is applicable to each compound by itself or to the combination or combinations of one or more compounds according to the invention as illustrated, for example, in the next immediate paragraph. The same understanding also applies to pharmaceutical composition(s) comprising such compound or compounds and method(s) of treatment involving such compound or compounds.

The compounds according to the invention can have pharmacological properties; in particular, the compounds according to the invention can be inhibitors of HCV protease, each compound by itself or one or more compounds according to the invention can be combined with one or more compounds selected from within the invention. The compound(s) can be useful for treating diseases such as, for example, HCV, HW, (AIDS, Acquired Immune Deficiency Syndrome), and related disorders, as well as for modulating the activity of hepatitis C virus (HCV) protease, preventing HCV, or ameliorating one or more symptoms of hepatitis C.

The compounds according to the invention may be used for the manufacture of a medicament to treat disorders associated with the HCV protease, for example, the method comprising bringing into intimate contact a compound according to the invention and a pharmaceutically acceptable carrier.

In another embodiment, this invention provides pharmaceutical compositions comprising the inventive compound or compounds as an active ingredient. The pharmaceutical compositions generally additionally comprise at least one pharmaceutically acceptable carrier diluent, excipient or carrier (collectively referred to herein as carrier materials). Because of their HCV inhibitory activity, such pharmaceutical compositions possess utility in treating hepatitis C and related disorders.

In yet another embodiment, the present invention discloses methods for preparing pharmaceutical compositions comprising the inventive compounds as an active ingredient. In the pharmaceutical compositions and methods of the present invention, the active ingredients will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition.

Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like.

Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. HCV inhibitory activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and pacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The compounds according to the invention may also be deliverable transdermally. The transdermal compositions may take the form of creams, lotions, aerosols and/or emulsions and can be included in a transdermal patch of the matrix or reservoir type as are conventional in the art for this purpose.

The compounds according to the invention may also be administered orally, intravenously, intranasally, intrathecally or subcutaneously.

The compounds according to the invention may also comprise preparations which are in a unit dosage form. In such form, the preparation is subdivided into suitably sized unit doses containing appropriate quantities of the active components, e.g., an effective amount to achieve the desired purpose.

The quantity of the inventive active composition in a unit dose of preparation may be generally varied or adjusted from about 1.0 milligram to about 1,000 milligrams, preferably from about 1.0 to about 950 milligrams, more preferably from about 1.0 to about 500 milligrams, and typically from about 1 to about 250 milligrams, according to the particular application. The actual dosage employed may be varied depending upon the patient's age, sex, weight and severity of the condition being treated. Such techniques are well known to those skilled in the art.

Generally, the human oral dosage form containing the active ingredients can be administered 1 or 2 times per day. The amount and frequency of the administration will be regulated according to the judgment of the attending clinician. A generally recommended daily dosage regimen for oral administration may range from about 1.0 milligram to about 1,000 milligrams per day, in single or divided doses.

Some useful terms are described below:

Capsule—refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the active ingredients with suitable diluents. The tablet can be prepared by compression of mixtures or granulations obtained by wet granulation, dry granulation or by compaction.

Oral gel—refers to the active ingredients dispersed or solubilized in a hydrophillic semi-solid matrix.

Powder for constitution refers to powder blends containing the active ingredients and suitable diluents which can be suspended in water or juices.

Diluent—refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

Disintegrant—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

Binder—refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d'l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glident—material that prevents caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Bioavailability—refers to the rate and extent to which the active drug ingredient or therapeutic moiety is absorbed into the systemic circulation from an administered dosage form as compared to a standard or control.

Conventional methods for preparing tablets are known. Such methods include dry methods such as direct compression and compression of granulation produced by compaction, or wet methods or other special procedures. Conventional methods for making other forms for administration such as, for example, capsules, suppositories and the like are also well known.

In yet another embodiment, the compositions of the invention may be used for the treatment of HCV in humans in combination with antiviral and/or immunomodulatory agents. Examples of such antiviral and/or immunomodulatory agents include intron, pegylated intron, ribavirin and the like. Illustrative examples include, but are not limited to, Ribavirin ((formula L, from Schering-Plough Corporation, Madison, New Jersey) and Levovirin™ (from ICN Pharmaceuticals, Costa Mesa, California), VP 50406™ (from Viropharma, Incorporated, Exton, Pennsylvania), ISIS14803™ (from ISIS Pharmaceuticals, Carlsbad, Calif.), Heptazyme™ (from Ribozyme Pharmaceuticals, Boulder, Colo.), VX 497™ (from Vertex Pharmaceuticals, Cambridge, Mass.), Thymosin™ (from SciClone Pharmaceuticals, San Mateo, California), Maxamine™ (Maxim Pharmaceuticals, San Diego, Calif.), mycophenolate mofetil (from Hoffman-LaRoche, Nutley, N.J.), interferon (such as, for example, interferon-alpha, PEG-interferon alpha conjugates) and the like. “PEG-interferon alpha conjugates” are interferon alpha molecules covalently attached to a PEG molecule. Illustrative PEG-interferon alpha conjugates include interferon alpha-2a (Roferon™, from Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™), interferon alpha-2c (Berofor Alpha™, from Boehringer Ingelheim, Ingelheim, Germany) or consensus interferon as defined by determination of a consensus sequence of naturally occurring interferon alphas (Infergen™, from Amgen, Thousand Oaks, California).

As stated earlier, the invention includes tautomers, rotamers, enantiomers and other stereoisomers of the inventive compounds also. Thus, as one skilled in the art appreciates, some of the inventive compounds may exist in suitable isomeric forms. Such variations are contemplated to be within the scope according to the invention.

Another embodiment according to the invention discloses a method of making the compounds disclosed herein. The compounds may be prepared by several techniques known in the art. Illustrative procedures are outlined in the following reaction schemes. The illustrations should not be construed to limit the scope according to the invention which is defined in the appended claims. Alternative mechanistic pathways and analogous structures will be apparent to those skilled in the art.

It is to be understood that while the following illustrative schemes describe the preparation of a few representative inventive compounds, suitable substitution of any of both the natural and unnatural amino acids will result in the formation of the desired compounds based on such substitution. Such variations are contemplated to be within the scope according to the invention.

For the procedures described below, the following abbreviations are used:

### Abbreviations

### THF: Tetrahydrofuran

### DMF: N,N-Dimethylformamide

EtOAc: Ethyl acetate
AcOH: Acetic acid

### NMM: N-Methylmorpholine

### DIAD: Diisopropylazodicarboxylate

### MeOH: Methanol

### EtOH: Ethanol

Et₂O: Diethyl ether

### DMSO: Dimethylsulfoxide

### HOBt: N-Hydroxybenzotriazole

### DCM: Dichloromethane

### DCC: 1,3-Dicyclohexylcarbodiimide

### Bn: Benzyl

### Bz: Benzyl

### Et: Ethyl

### Ph: Phenyl

iBoc: isobutoxycarbonyl
iPr: isopropyl
^{t}Bu or Bu^{t}: tert-Butyl
Boc: tert-Butyloxycarbonyl

### Cbz: Benzyloxycarbonyl

### Cp: Cylcopentyldienyl

Ts: p-toluenesulfonyl

### Me: Methyl

Ms or Mesyl: Methane sulfonyl
HATU: O-(7-azabenzotriazol-1-yl)-1,1,3,3-tetramethyluronium hexafluorophosphate

### DMAP: 4-N,N-Dimethylaminopyridine

Bop: Benzotriazol-1-yl-oxy-tris(dimethylamino)hexafluorophosphate

### PCC: Pyridiniumchlorochromate

DIBAL-H: diisopropyl aluminum hydride
rt or RT: Room temperature
quant.: Quantitative yield
h or hr: hour
min: minute
TFA: Trifluoroacetic acid

### TLC: Thin Layer Chromatography

### Aq.: Aqueous

Kᵢ: inhibition constant
Sat'd: saturated

### TFE: Trifluoroethanol

pTSA: paratoluenesulfonic acid

### HPLC: High Performance Liquid Chromatography

PAP: 4-phenylazophenol
HMC: 7-hydroxy-4-methyl-coumarin
Np: nitrophenol
DIT: dithiothreitol
MOPS: 3-[N-Morpholino]propanesulfonic acid
TBTU: 2-(1H-benzotriazol-1-yl)-1,1,3,3-tetramethyluronium tetrafluoroborate

### General Schemes for Preparation of Target Compounds

### Procedure for the Synthesis of Compound 1

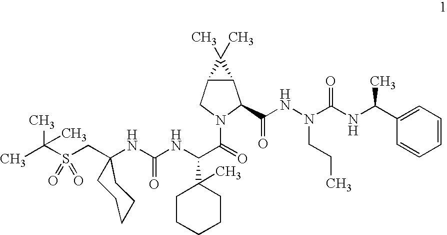

### Step A:

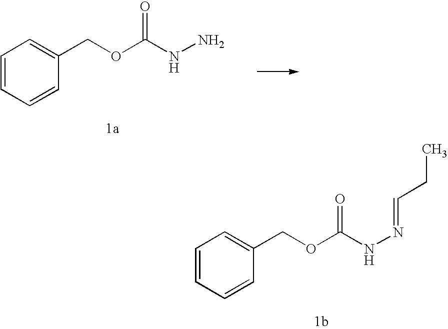

A solution of Cbz-carbazate 1a (6.00 g, 36.11 mmol) in toluene (140 mL) was treated with propinaldehyde (2.4 g, 41.58 mmol) and stirred at 70° C. for 2 h and it. for 12 h. The reaction mixture was concentrated in vacuo and used as it is in the following step. 8g of colorless solid 1b was isolated.

### Step B:

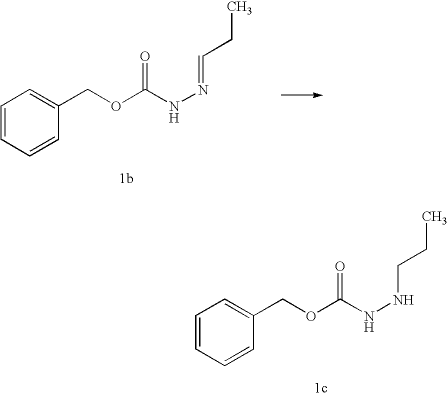

A solution of hydrazone 1b (1.5 g, 6.89 mmol), sodium cyanoborohydride (435 mg, 6.89 mmol) in THF (30 mL) was cooled to 0° C. and treated with p-toluenesulfonic acid in THF dropwise. After completion of the reaction (indicated by TLC), the reaction mixture was diluted with aq. NaOH (1 M) and extracted into EtOAc. The combined organic were dried with MgSO₄, filtered concentrated in vacuo and used as it is in the next step.

### Step C:

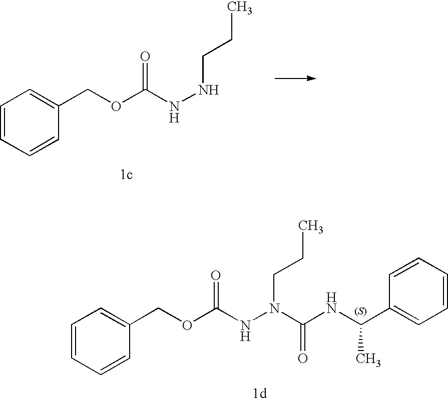

A solution of reduced hydrazine in methylene chloride was treated with (5)-methylbenzyl isocyanate and stirred at rt. for 3 h. The reaction mixture was concentrated in vacuo and purified by chromatography (acetone/hexanes 0/1->1:2) to yield pure product.

### Step D:

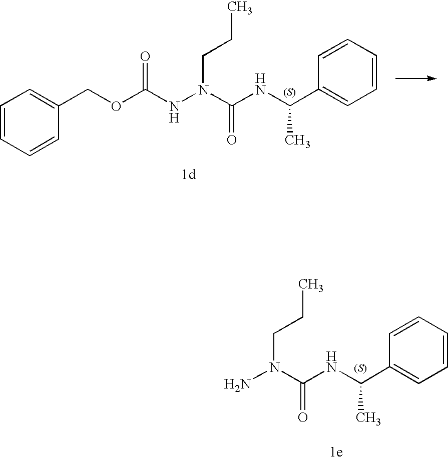

A solution of Cbz protected hydrazide (1d, 800 mg, 2.25 mmol) in ethyl acetate (30 mL) was treated with Pd/C (10%) and hydrogenated at 15-20 psi for 2 h. The reaction mixture was filtered through a plug of Celite® and concentrated in vacuo. The residue was used as it is without further purification.

### Step E:

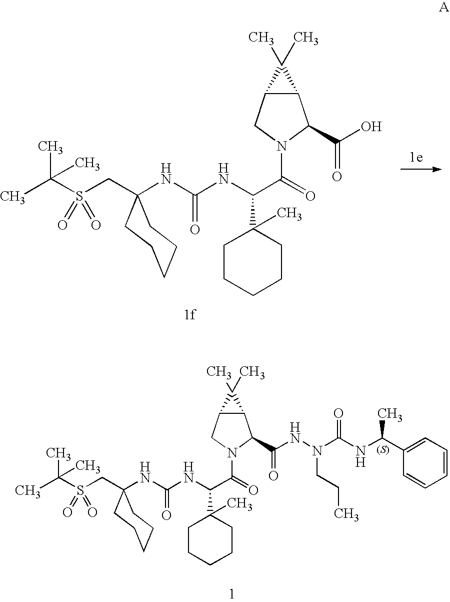

solution of acid (1f, 100 mg, 0.18 mmol) and amine (1e, 39 mg, 0.18 mmol) in CH₂Cl₂ and DMF (4 mL, 1:1) was cooled to 0° C. and treated with TBTU (96 mg, 0.30 mmol) and NMM (72 mg, 0.72 mmol) and stirred at rt. for 48 h. The reaction mixture was concentrated in vacuo and the residue diluted with aq. HCl (1 M soln. 40 mL). The reaction mixture was extracted with ethyl acetate (100 mL). The combined organic layers were washed with aq. satd. sodium bicarbonate, brine, dried (MgSO₄) filtered, concentrated in vacuo and purified by chromatography to yield pure product.

### Procedure for the Synthesis of Compound 2.

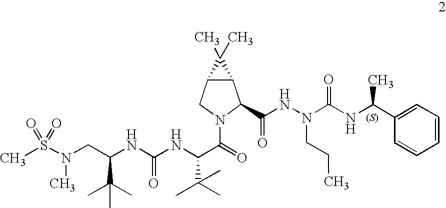

### Step A:

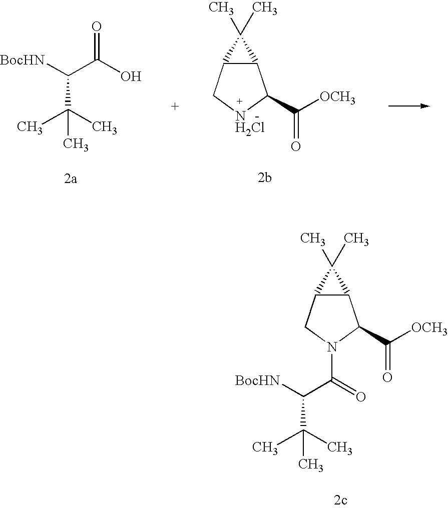

The amino ester 2b was prepared following the method of R. Zhang and J. S. Madalengoitia (J. Org. Chem. 1999, 64, 330), with the exception that the Boc group was cleaved by the reaction of the Boc-protected amino acid with methanolic HCl

(Note: In a variation of the reported synthesis, the sulfonium ylide used to install the dimethylcyclopropyl ring was replaced with the corresponding phosphonium ylide)

A solution of Boc-tert-Leu 2a (Fluka, 5.0 g 21.6 mmol) in dry CH₂Cl₂/DMF (50 mL, 1:1) was cooled to 0° C. and treated with the amine 2b (5.3 g, 25.7 mmol), NMM (6.5 g, 64.8 mmol) and BOP reagent (11.6 g, 25.7 mmol). The reaction was stirred at rt. for 24 hrs, diluted with aq. HCl (1 M) and extracted with CH₂Cl₂. The combined organic layers were washed with HCl (aq, 1 M), sat'd. NaHCO₃, brine, dried (MgSO₄), filtered and concentrated in vacuo and purified by chromatography (SiO₂, acetone/Hexane 1:5) to yield 2c as a colorless solid.

### Step B:

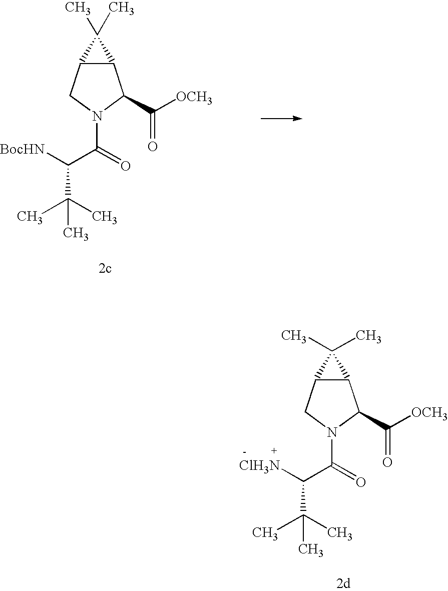

A solution of methyl ester 2c (4.0 g, 10.46 mmol) was dissolved in HCl (4 M soln. dioxane) and stirred at rt. for 3 h. The reaction mixture was concentrated in vacuo to obtain the amine hydrochloride salt used in the next step without further purification.

### Step C:

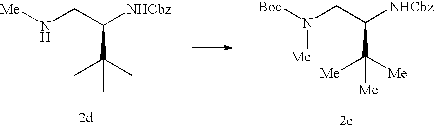

A solution of amine 2d* (4.0 g, 15.14 mmol) in CH₂Cl₂ (100 mL) was treated with di-tert-butyldicarbonate (4.13 g, 18.91 mmol) and stirred at rt. for 12 h. The reaction mixture was concentrated in vacuo and purified by chromatography (SiO₂, EtOAc/Hexanes 1:5) to yield 2e. *Obtained by Cbz protection of tert-Leu-NH—CH₃ (TCI, Jpn) followed by reduction with BH₃.DMS

### Step D:

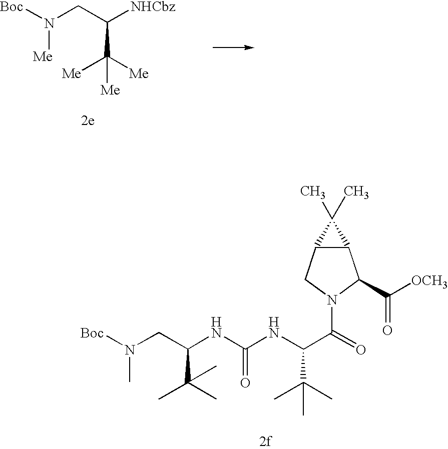

A solution of 2e (2.3 g, 6.31 mmol) in methanol was treated with Pd(OH)₂/C (886 mg) and hydrogenated for 3 h in a parr shaker. The reaction mixture was filtered through a plug of celite and used as it is in the next step (1.3 g).

A solution of deprotected amine (2.6 g, 11.3 mmol) was taken in dry CH₂Cl₂ and cooled to 0° C. and treated with 4-nitrophenylcarbamate of 2d. The reaction mixture was stirred for 48 h at rt. The reaction mixture was further diluted with dichloromethane and washed with aq. saturated NaHCO₃ and brine. The organic layer was concentrated in vacuo and purified by chromatography to yield 2f (4.42 g, 72%)

### Step E:

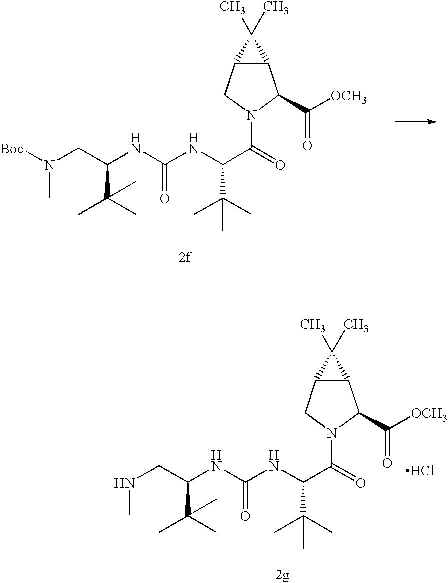

A solution of 2f (430 mg, 0.8 mmol) in 4 M HCl in dioxane was stirred at rt. for 1 h and concentrated in vacuo. The residue 2g (380 mg) was used as it is in the next step without purification.

### Step E1:

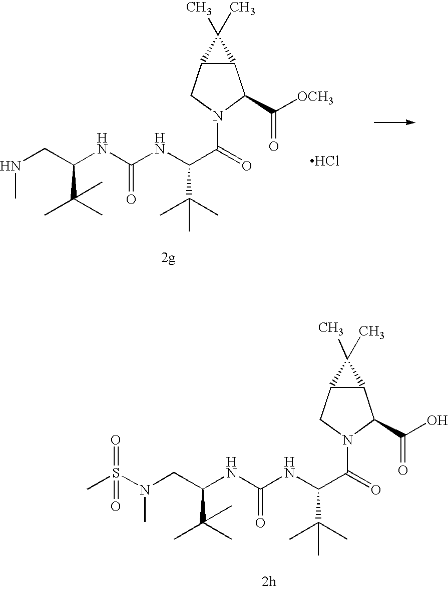

A solution amine salt 2g (172 mg, 0.36 mmol) was dissolved in CH₂Cl₂ and cooled to 0° C. The reaction mixture was treated with Et₃N (54 mg, 0.53 mmol) and methanesulfonyl chloride (61 mg, 0.53 mmol) and stirred at rt. overnight. The reaction mixture was washed with 1 M aq HCl, and the organic layer was extracted with CH₂Cl₂. The organic layer was dried with MgSO₄, filtered, and concentrated in vacuo. The crude product was purified by chromatography (20% to 50% acetone/hexanes) to yield precursor of 2h 90 mg).

Methyl ester was dissolved in THF and H₂O (approximately 3:1 ratio) and treated with LiOH.H₂O (18 mg). The reaction mixture was treated with MeOH until homogeneous. The reaction mixture was stirred at rt. for approximately 3 hr, treated with 1 M aq HCl and concentrated in vacuo. The aqueous layer was extracted with CH₂Cl₂, dried with MgSO₄, filtered, and concentrated in vacuo to yield 2h as a colorless solid.

### Step E2:

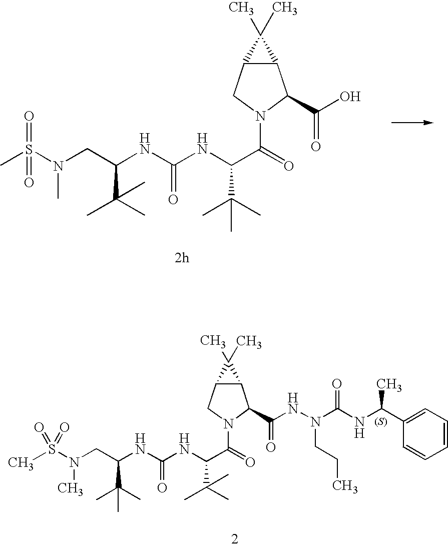

A solution of acid 2h (100 mg, 0.2 mmol) in CH₂Cl₂ and DMF (4 mL, 1:1) was cooled to 0° C. and treated with 1e (39 mg, 0.18 mmol), HATU (114 mg, 0.30 mmol) and NMM (72 mg, 0.72 mmol) and stirred at rt. for 12 h. The reaction mixture was concentrated in vacuo and the residue diluted with aq. HCl (1 M soln. 40 mL) The reaction mixture was extracted with ethylacetate (100 mL). The combined organic layers were washed with aq. satd. sodium bicarbonate, brine, dried (MgSO₄) filtered, concentrated in vacuo and purified by chromatography to yield pure product 2 as a colorless solid.

### Procedure for the Synthesis of Compound 3.

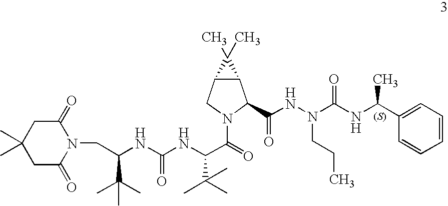

### Step A:

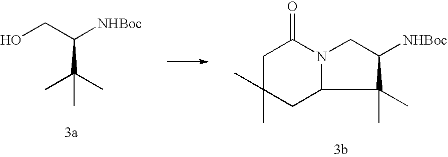

A solution of the alcohol 3a (1.00 g, 4.6 mmol) in anhydrous CH₂Cl₂ (30 mL) in an inert atmosphere was treated with triphenylphosphine (1.52 g, 5.75 mmol) and dimethylglutarimide (780 mg, 5.52 mmol). The reaction mixture was cooled to 0° C. and treated with DIAD (930 mg, 4.60 mmol, in 4 mL CH₂Cl₂) dropwise and warmed to rt. It was stirred at rt. for 5 h and concentrated in vacuo. The residue was purified by chromatography (SiO₂, Hexanes/acetone 1:0->1:1) to obtained 3b as a colorless solid

### Step B:

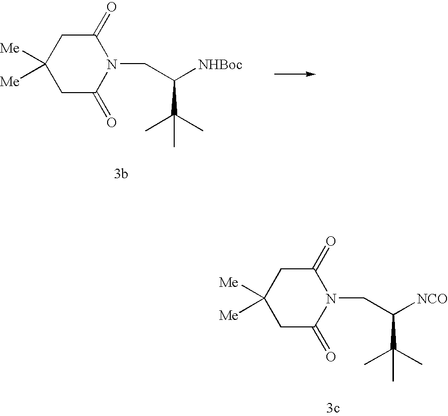

A solution of 3b (500 mg, 1.5 mmol) in HCl (15 mL, 4M soln. in dioxane) was stirred at rt. for 1 h and concentrated in vacuo. The residue was used in further reaction without purification. A solution of the deprotected amine in CH₂Cl₂ (10 mL) aq. saturated NaHCO₃ (10 mL) at 0° C. was treated with phosgene (5 mL, 15% soln. in toluene) and stirred at 0° C. for 2 h. The reaction mixture was diluted with CH₂Cl₂ (50 mL) and the organic layer was washed with cold aq. NaHCO₃. The organic layer was dried (MgSO₄) filtered and further diluted with 3 mL toluene, concentrated the methylene chloride layer and used as a solution of 3c.

### Step C:

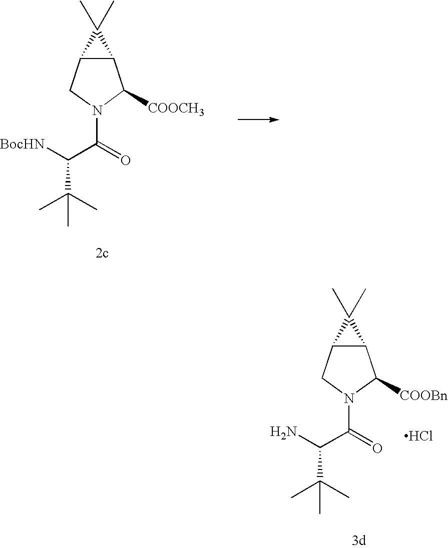

2c (2 g, 5.2 mmol) was dissolved in THF and H₂O (3:1) and treated with LiOH.H₂O (658 mg, 15.7 mmol). The reaction mixture was treated with MeOH until it turned homogeneous. The reaction mixture was stirred at rt for approximately 2 hr. The reaction mixture was treated with 1 M aq HCl and concentrated in vacuo. The residue was diluted with water and extracted with CH₂Cl₂. The combined organic layers were dried with MgSO₄, filtered, and concentrated in vacuo to yield acid directly used in the following step without purification.

The acid (2.1 g, 5.7 mmol) was dissolved in DMF and cooled to 0° C. The reaction mixture was treated with cesium carbonate (2.2 g, 6.8 mmol) and benzyl bromide (1.2 g, 6.8 mmol) and stirred at rt overnight. The reaction mixture was concentrated under vacuum, and the residue was diluted with H₂O. The aqueous layer was extracted with EtOAc and the combined organic layer was washed with H₂O, dried with MgSO₄, filtered, and concentrated in vacuo. The crude product was purified using silica gel chromatography (0%--->15% EtOAc/hexanes) to yield benzyl ester which was deprotected with 4 M HCl in dioxane to yield 3d as a colorless solid used in next step.

### Step D:

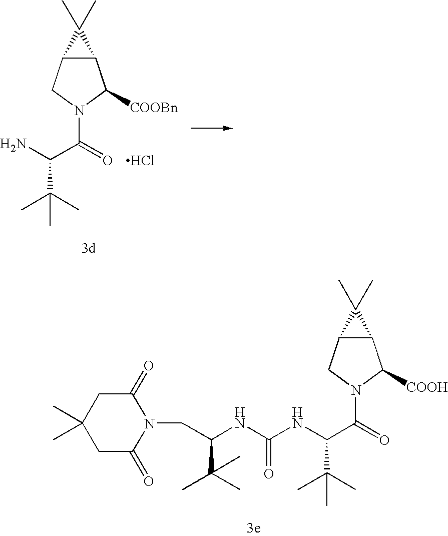

3d (800 mg, 2.00 mmol) was dissolved in anhydrous DCM and cooled to 0° C. The reaction mixture was treated with NMM, stirred for 5 minutes, and treated a 0.5 M solution of the isocyanate 3c. The reaction mixture was stirred at rt overnight. The reaction mixture was diluted with water and extracted with CH₂Cl₂. The combined organic layer was washed with 1M aq HCl and saturated NaHCO₃, dried with MgSO₄, filtered, and concentrated in vacuo. The crude product was purified using silica gel chromatography with (0%-->50% EtOAc/hexanes) to yield benzyl ester of 3e which was dissolved in methanol and treated with Pd/C (10%) and hydrogenated for 3 h. The reaction mixture was filtered through a plug of celite and concentrated in vacuo to yield 3e as a colorless solid.

### Step E:

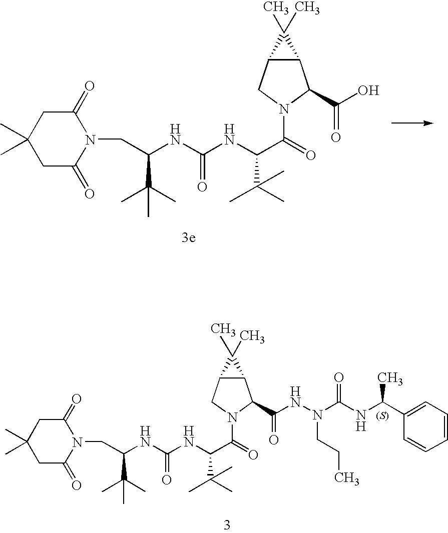

A solution of acid (100 mg, 0.18 mmol) in CH₂Cl₂ and DMF (4 mL, 1:1) was cooled to 0° C. and treated with HATU (114 mg, 0.30 mmol) and NMM (72 mg, 0.72 mmol) and stirred at rt. for 12 h. The reaction mixture was concentrated in vacuo and the residue diluted with aq. HCl (1 M soln. 40 mL) The reaction mixture was extracted with ethylacetate (100 mL). The combined organic layers were washed with aq. satd. sodium bicarbonate, brine, dried (MgSO₄) filtered, concentrated in vacuo and purified by chromatography to yield 3 as a colorless solid.

### Procedure for the Synthesis of Compound 4

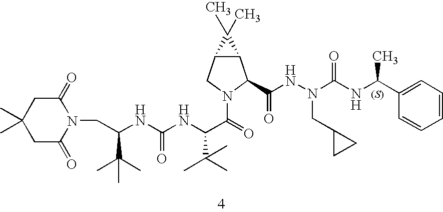

### Step A:

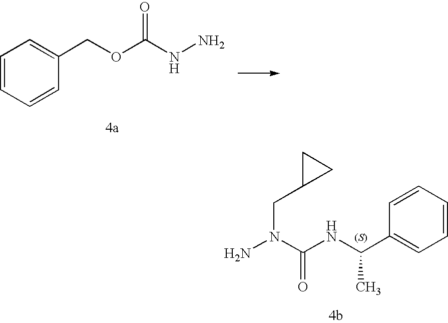

The synthesis of 4b from 4a was identical to synthesis of 1e in preparation of compound 1 from Step A through Step D. Proponal was replaced with cyclopropylacetaldehyde.

### Step B:

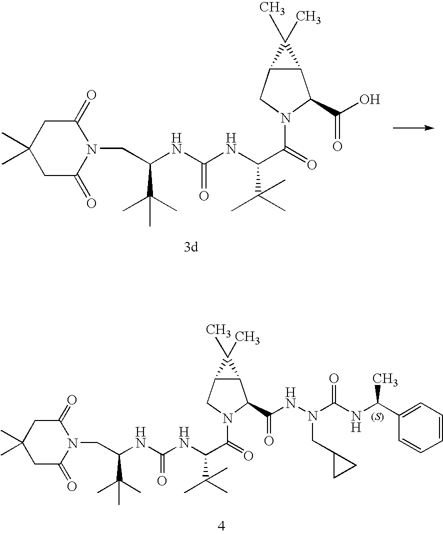

A solution of acid 3d (100 mg, 0.18 mmol) and amine 4b (100 mg, 0.4 mmol) in CH₂Cl₂ and DMF (4 mL, 1:1) was cooled to 0° C. and treated with HATU (96 mg, 0.30 mmol) and NMM (72 mg, 0.72 mmol) and stirred at rt. for 12 h. The reaction mixture was concentrated in vacuo and the residue diluted with aq. HCl (1 M soln. 40 mL) The reaction mixture was extracted with ethylacetate (100 mL). The combined organic layers were washed with aq. satd. sodium bicarbonate, brine, dried (MgSO₄) filtered, concentrated in vacuo and purified by chromatography (acetone/hexanes 0:1-2:1) to yield 4 pure product.

Procedure for the synthesis of compound 5

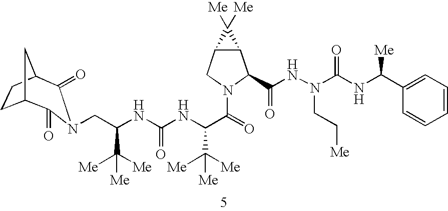

### Step A:

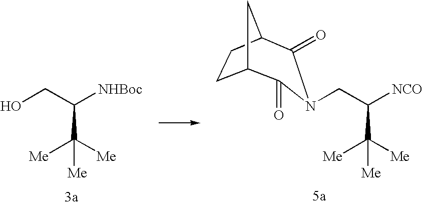

The synthesis of isocyanate 5a was accomplished following similar synthetic route described for isocynate 3b except 3-aza-bicyclo[3.2.1]octane-2,4-dione was used in the place of 4,4-dimethyl-piperidine-2,6-dione. The isocyante 5a was used as a solution in methylene chloride and toluene.

### Step B:

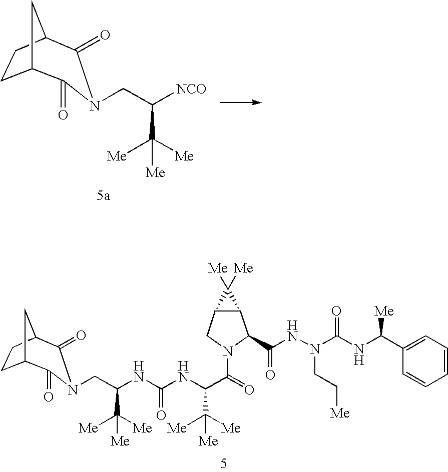

The conversion of isocyante 5a to 5 was similar to the procedure described for preparation of compound 3 following Steps C through Step E.

The present invention relates to novel HCV protease inhibitors. This utility can be manifested in their ability to inhibit the HCV NS3/NS4a serine protease. A general procedure for such demonstration is illustrated by the following in vitro assay.

### Assay for HCV Protease Inhibitory Activity:

Spectrophotometric Assay Spectrophotometric assay for the HCV serine protease can be performed on the inventive compounds by following the procedure described by R. Zhang et al, Analytical Biochemistry, 270 (1999) 268-275, the disclosure of which is incorporated herein by reference. The assay based on the proteolysis of chromogenic ester substrates is suitable for the continuous monitoring of HCV NS3 protease activity. The substrates are derived from the P side of the NS5A-NS5B junction sequence (Ac-DTEDVVX(Nva), where X=A or P) whose C-terminal carboxyl groups are esterified with one of four different chromophoric alcohols (3- or 4-nitrophenol, 7-hydroxy-4-methyl-coumarin, or 4-phenylazophenol). Illustrated below are the synthesis, characterization and application of these novel spectrophotometric ester substrates to high throughput screening and detailed kinetic evaluation of HCV NS3 protease inhibitors.

### Materials and Methods:

Materials: Chemical reagents for assay related buffers are obtained from Sigma Chemical Company (St. Louis, Mo.). Reagents for peptide synthesis were from Aldrich Chemicals, Novabiochem (San Diego, Calif.), Applied Biosystems (Foster City, Calif.) and Perseptive Biosystems (Framingham, Massachusetts). Peptides are synthesized manually or on an automated ABI model 431A synthesizer (from Applied Biosystems). UV/VIS Spectrometer model LAMBDA 12 was from Perkin Elmer (Norwalk, Conn.) and 96-well UV plates were obtained from Corning (Corning, N.Y.). The prewarming block can be from USA Scientific (Ocala, Florida) and the 96-well plate vortexer is from Labline Instruments (Melrose Park, Illinois). A Spectramax Plus microtiter plate reader with monochrometer is obtained from Molecular Devices (Sunnyvale, Calif.).

Enzyme Preparation: Recombinant heterodimeric HCV NS3/NS4A protease (strain 1a) is prepared by using the procedures published previously (D. L. SalI et al, Biochemistry, 37 (1998) 3392-3401). Protein concentrations are determined by the Biorad dye method using recombinant HCV protease standards previously quantified by amino acid analysis. Prior to assay initiation, the enzyme storage buffer (50 mM sodium phosphate pH 8.0, 300 mM NaCl, 10% glycerol, 0.05% lauryl maltoside and 10 mM DTT) is exchanged for the assay buffer (25 mM MOPS pH 6.5, 300 mM NaCl, 10% glycerol, 0.05% lauryl maltoside, 5 μM EDTA and 5 μM DTT) utilizing a Biorad Bio-Spin P-6 prepacked column.

Substrate Synthesis and Purification: The synthesis of the substrates is done as reported by R. Zhang et al, (ibid.) and is initiated by anchoring Fmoc-Nva-OH to 2-chlorotrityl chloride resin using a standard protocol (K. Barbs et al, Int. J. Pept. Protein Res., 37 (1991), 513-520). The peptides are subsequently assembled, using Fmoc chemistry, either manually or on an automatic ABI model 431 peptide synthesizer. The N-acetylated and fully protected peptide fragments are cleaved from the resin either by 10% acetic acid (HOAc) and 10% trifluoroethanol (TFE) in dichloromethane (DCM) for 30 min, or by 2% trifluoroacetic acid (TFA) in DCM for 10 mM. The combined filtrate and DCM wash is evaporated azeotropically (or repeatedly extracted by aqueous Na₂CO₃ solution) to remove the acid used in cleavage. The DCM phase is dried over Na₂SO₄ and evaporated.

The ester substrates are assembled using standard acid-alcohol coupling procedures (K. Holmber et al, Acta Chem. Scand., B33 (1979) 410-412). Peptide fragments are dissolved in anhydrous pyridine (30-60 mg/ml) to which 10 molar equivalents of chromophore and a catalytic amount (0.1 eq.) of para-toluenesulfonic acid (pTSA) were added. Dicyclohexylcarbodiimide (DCC, 3 eq.) is added to initiate the coupling reactions. Product formation is monitored by HPLC and can be found to be complete following 12-72 hour reaction at room temperature. Pyridine solvent is evaporated under vacuum and further removed by azeotropic evaporation with toluene. The peptide ester is deprotected with 95% TFA in DCM for two hours and extracted three times with anhydrous ethyl ether to remove excess chromophore. The deprotected substrate is purified by reversed phase HPLC on a C3 or C8 column with a 30% to 60% acetonitrile gradient (using six column volumes). The overall yield following HPLC purification can be approximately 20-30%. The molecular mass can be confirmed by electrospray ionization mass spectroscopy. The substrates are stored in dry powder form under desiccation.

Spectra of Substrates and Products: Spectra of substrates and the corresponding chromophore products are obtained in the pH 6.5 assay buffer. Extinction coefficients are determined at the optimal off-peak wavelength in 1-cm cuvettes (340 rim for 3-Np and HMC, 370 nm for PAP and 400 nm for 4-Np) using multiple dilutions. The optimal off-peak wavelength is defined as that wavelength yielding the maximum fractional difference in absorbance between substrate and product (product OD−substrate OD)/substrate OD).

Protease Assay: HCV protease assays are performed at 30° C. using a 200 μl reaction mix in a 96-well microtiter plate. Assay buffer conditions (25 mM MOPS pH 6.5, 300 mM NaCl, 10% glycerol, 0.05% lauryl maltoside, 5 μM EDTA and 5 μM DTT) are optimized for the NS3/NS4A heterodimer (D. L. SalI et al, ibid.)). Typically, 150 μl mixtures of buffer, substrate and inhibitor are placed in wells (final concentration of DMSO≦4% v/v) and allowed to preincubate at 30° C. for approximately 3 minutes. Fifty μls of prewarmed protease (12 nM, 30° C.) in assay buffer, is then used to initiate the reaction (final volume 200 μl).The plates are monitored over the length of the assay (60 minutes) for change in absorbance at the appropriate wavelength (340 nm for 3-Np and HMC, 370 nm for PAP, and 400 nm for 4-Np) using a Spectromax Plus microtiter plate reader equipped with a monochrometer (acceptable results can be obtained with plate readers that utilize cutoff filters). Proteolytic cleavage of the ester linkage between the Nva and the chromophore is monitored at the appropriate wavelength against a no enzyme blank as a control for non-enzymatic hydrolysis. The evaluation of substrate kinetic parameters is performed over a 30-fold substrate concentration range (˜6-200 μM). Initial velocities are determined using linear regression and kinetic constants are obtained by fitting the data to the Michaelis-Menten equation using non-linear regression analysis (Mac Curve Fit 1.1, K. Raner). Turnover numbers (k_{cat}) are calculated assuming the enzyme is fully active.

Evaluation of Inhibitors and Inactivators: The inhibition constants (Ki) for the competitive inhibitors Ac-D-(D-Gla)-L-1-(Cha)-C—OH (27), Ac-DTEDVVA(Nva)-OH and Ac-DTEDVVP(Nva)-OH are determined experimentally at fixed concentrations of enzyme and substrate by plotting vₒ/vᵢ vs. inhibitor concentration ([I]ₒ) according to the rearranged Michaelis-Menten equation for competitive inhibition kinetics: vₒ/vᵢ=1+[I]ₒ/(Kᵢ(1+[S]ₒ/Kₘ)), where vₒ is the uninhibited initial velocity, vᵢ is the initial velocity in the presence of inhibitor at any given inhibitor concentration ([I]ₒ) and [S]ₒ is the substrate concentration used. The resulting data are fitted using linear regression and the resulting slope, 1/(Kᵢ(1+[S]ₒ/Kₘ), is used to calculate the Kᵢ value. The obtained Ki and IC₅₀ values (in nanoMolar) for some of the inventive compounds are shown below in Table 2.

The ranges of Ki and IC₅₀ values are as follows:

### A=Kᵢ<200 nM; B=Kᵢ>200 nM and <500 nM; C=Kᵢ>500 nM

### A=IC₅₀≦500 nM; B=IC₅₀>500 nM and ≦3000 nM

TABLE 2

| | | | IC₅₀ |
|---|---|---|---|
| Cmpd. | | Kᵢ | (replicon) |
| No. | Structure | nM | nM |
| 1 | 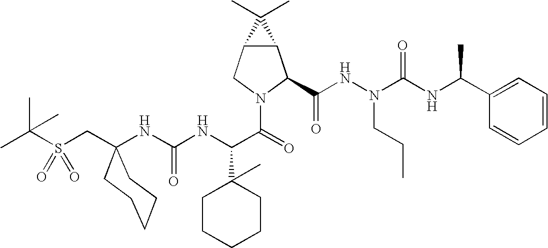 | A | B |
| 2 | 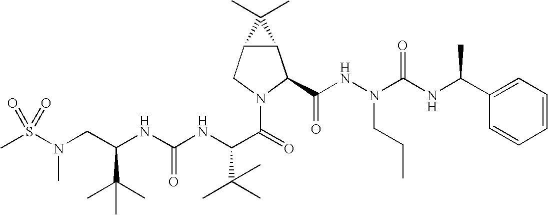 | A | A |
| 3 | 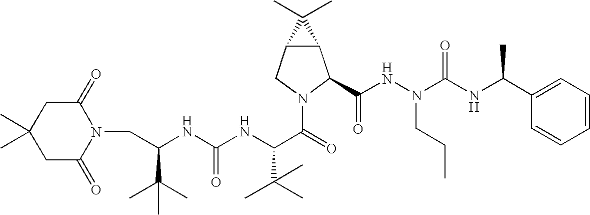 | A | A |
| 4 | 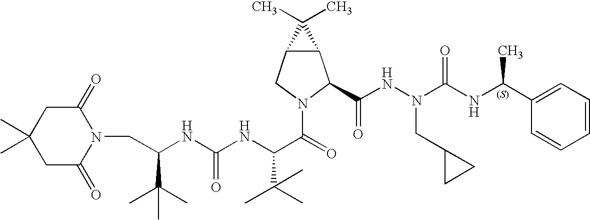 | B | NT |
| 5 | 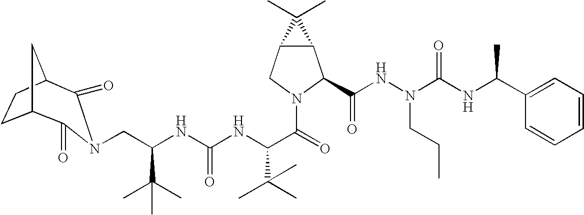 | A | A |
| 6 | 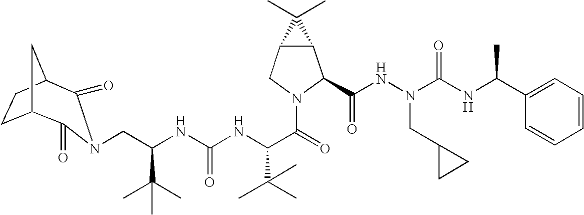 | B | B |
| 7 | 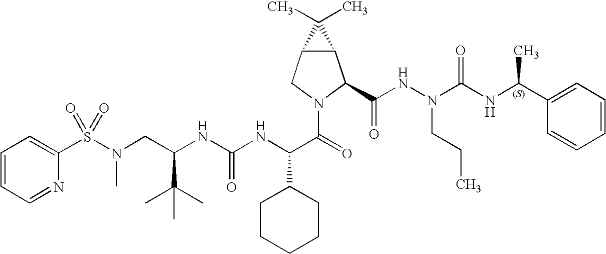 | B | NT |
| 8 | 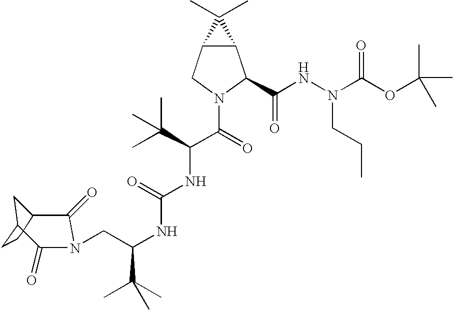 | C | |
| 9 | 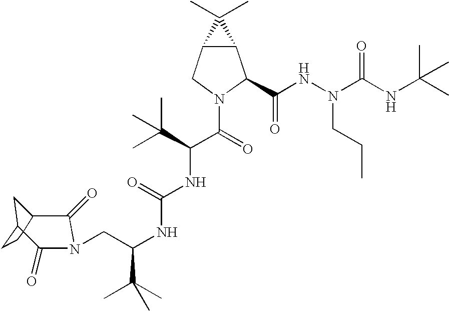 | C | |
| 10 | 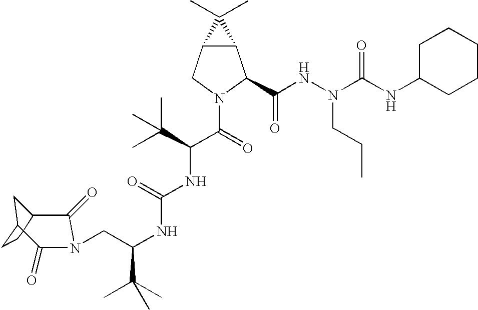 | C | |
| 11 | 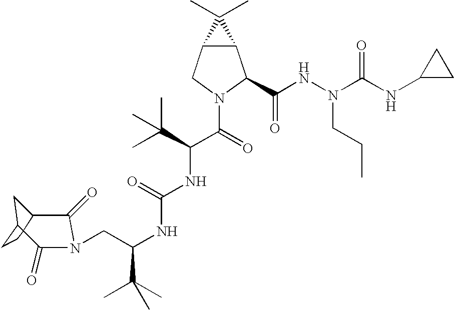 | C | |
| 12 | 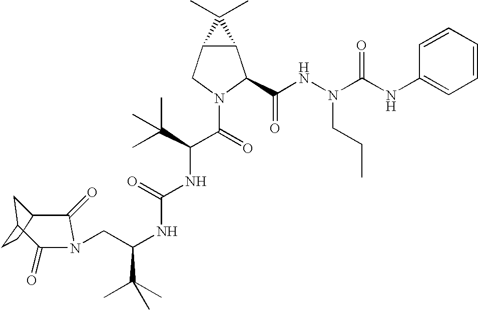 | C | |
| 13 | 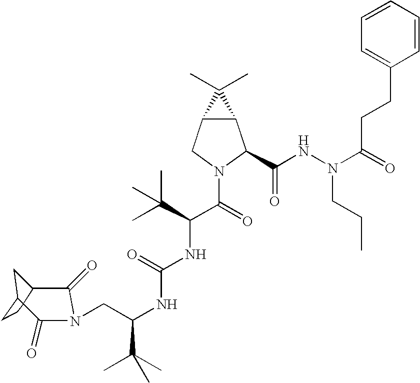 | C | |
| 14 | 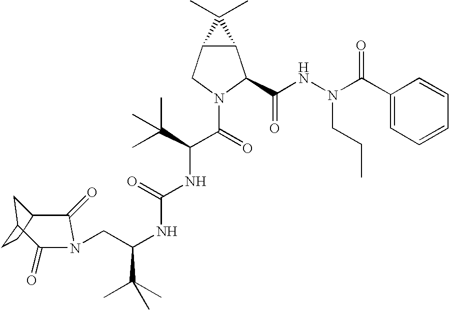 | C | |
| 15 | 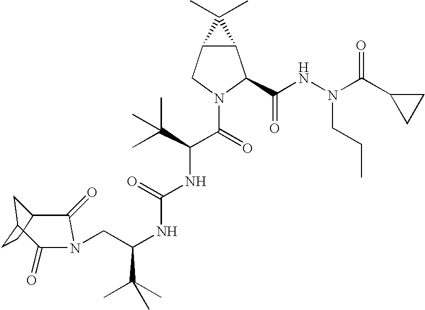 | C | |
| 16 | 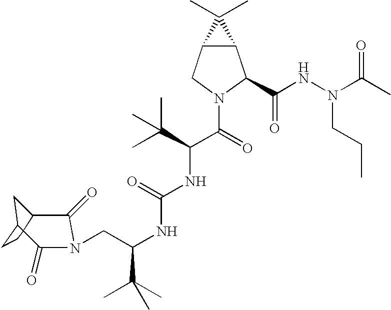 | C | |

NT = not tested

What is claimed is:

1. A compound, or enantiomer, stereoisomer, rotamer, tautomer, and racemate of said compound, or a pharmaceutically acceptable salt, solvate or ester of said compound, said compound having the general structure shown in Formula I: 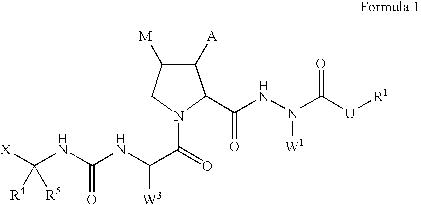
wherein:
R¹ is hydrogen, alkyl, alkenyl, alkynyl, aryl, arylalkyl, arylalkenyl, heteroalkyl, cycloalkenyl, cycloalkenylalkyl-, cycloalkenylalkenyl-, cycloalkyl, cycloalkylalkyl-,
cycloalkylalkenyl-, 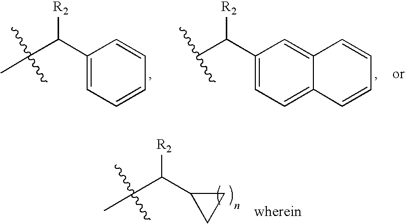
each of said , alkyl, alkenyl, alkynyl, aryl, arylalkyl, arylalkenyl, heteroalkyl, cycloalkenyl, cycloalkenylalkyl-, cycloalkenylalkenyl-, cycloalkyl, cycloalkylalkyl-,
cycloalkylalkenyl-, 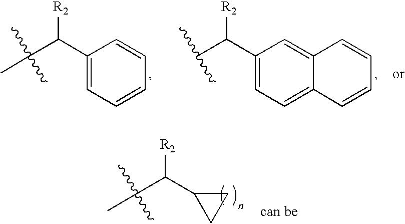
unsubstituted or substituted with one or moieties, which can be the same or different, each moiety being independently selected from the group consisting of halogen, nitro, alkyl, aminoalkyl, alkoxyalkyl-, aminoalkloxyalkyl-, alkenyl, heteroaryl, cycloalkyl, cycloalkenyl, heterocycloalkyl, heterocycloalkenyl, aryl, trihaloalkyl, dihaloalkyl, monohaloalkyl, alkylsulfonyl, and arylsulfonyl,
further wherein R² is selected from the group consisting of hydrogen, alkyl, cycloalkyl, alkenyl, alkynyl, cycloalkenyl, heteroalkyl, heterocycloalkyl, heterocycloalkenyl, aryl, heteroaryl,
cycloalkylalkyl-, cycloalkenylalkyl-, cycloalkylalkenyl-, cycloalkenylalkenyl-, heterocycloalkylalkyl-, heterocycloalkenylalkyl-, heterocycloalkylalkenyl-, heterocycloalkenylalkenyl-, arylalkyl, heteroaryl, and heteroarylalkyl-;
A and M are connected to each other such that the moiety: 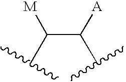
shown above in Formula I, forms either a three, four, five, six, seven or eight-membered cycloalkyl, a three, five, four, six, seven or eight-membered cycloalkenyl, a four to eight-membered heterocyclyl, a four to eight-membered heterocycloalkenyl, a six to ten-membered aryl, or a five to ten-membered heteroaryl wherein each of said three, four, five, six, seven or eight-membered cycloalkyl, three, four, five, six, seven or eight-membered cycloalkenyl, four to eight-membered heterocyclyl, four to eight-membered heterocycloalkenyl, six to ten-membered aryl, or five to ten-membered heteroaryl can be unsubstituted or substituted with one or more moieties, which can be the same or different, each moiety being independently selected from the group consisting of alkyl, alkenyl, alkynyl, halogen, trihaloalkyl, dihaloalkyl, monohaloalkyl, heteroalkyl, amino, aminoalkyl, alkoxyalkyl-, alkylsulfonyl-, and arylsulfonyl-;
X is selected from the group consisting of: 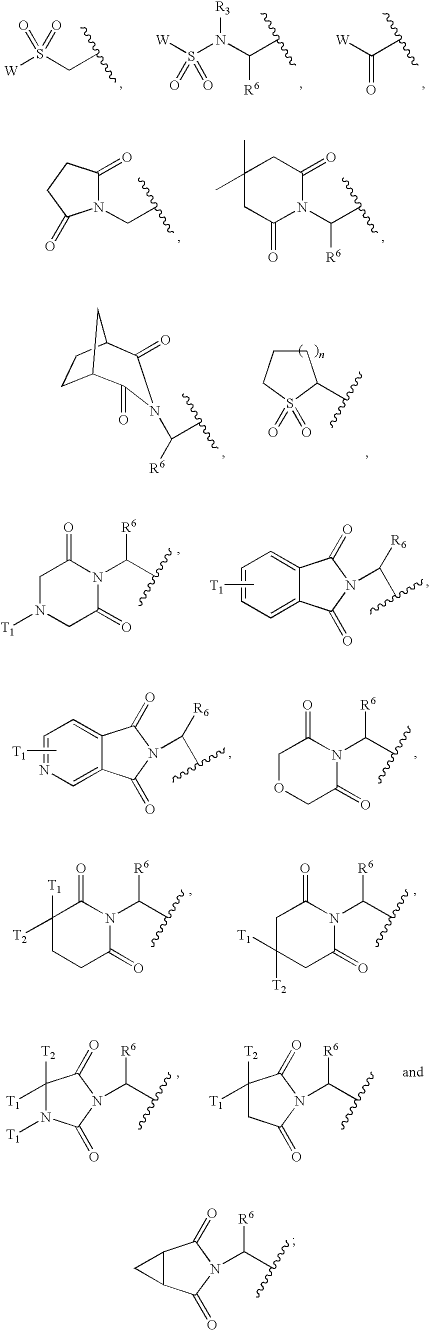
where T₁ and T₂ can be the same or different, each being independently selected from alkyl, aryl, heteroalkyl, heteroaryl, halo, amino, alkylamino-, alkylthio-, amido or carbamate urea;
W³ and R⁶ can be one or two moieties;
W is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
W¹ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
W³ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
R³ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
R⁴ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
R⁵ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
R⁶ is selected from the group consisting of H, alkyl-, alkenyl-, alkynyl-, cycloalkyl-, cycloalkenyl-, heteroalkyl-, heterocyclyl-, heterocycloalkenyl-, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, heteroarylalkenyl-, alkoxy, aryloxy, alkylthio, arylthio, amino, hydroxyl, amido, ester, carboxylic acid, carbamate, urea, ketone, aldehyde, cyano, nitro, halogen, alkylaryl, alkylheteroaryl-, alkenylaryl-, and alkenylheteroaryl-, wherein each of said alkyl-, alkenyl-, alkynyl, cycloalkyl-, cycloalkenyl-, heteroalkyl-, heterocyclyl-, heterocycloalkenyl, aryl-, heteroaryl-, cycloalkylalkyl-, cycloalkenylalkyl-, cycloalkylalkenyl-, cycloalkenylalkenyl-, heterocyclylalkyl-, heterocyclylalkenyl-, heterocycloalkenylalkyl-, heterocycloalkenylalkenyl-, arylalkyl-, arylalkenyl-, heteroarylalkyl-, and heteroarylalkenyl- can be unsubstituted or substituted with one or more moieties, which moieties can be the same or different, each moiety being independently selected from the group consisting of alkyl, alkenyl, alkynyl, monohaloalkyl, dihaloalkyl, trihaloalkyl, halogen aryl, arylalkyl, cycloalkyl, heterocycloalkyl, hydroxyl, thio, alkoxy, aryloxy, alkylthio, arylthio, amino, amido, ester, carboxylic acid, carbamate, urea, ketone, aldehyde, cyano, nitro, sulfamido, sulfoxide, sulfone, sulfonylurea, hydrazide, and hydroxamate;
or R⁴ and R⁵ together with the carbon to which they are attached form either a three to eight-membered cycloalkyl, a four to eight-membered heterocyclyl, three to eight-membered cycloalkenyl, a four to eight-membered heterocycloalkenyl, a six to ten membered aryl, or a five to ten-membered heteroaryl, wherein each of said three to eight-membered cycloalkyl, four to eight-membered heterocyclyl, three to eight-membered cycloalkenyl, four to eight-membered heterocycloalkenyl, six to ten membered aryl, or five to ten-membered heteroaryl can be unsubstituted or substituted with one or more moieties, which can be the same or different, each moiety being independently selected from the group consisting of alkyl, alkenyl, alkynyl, monohaloalkyl, dihaloalkyl, trihaloalkyl and halogen; or the moiety: 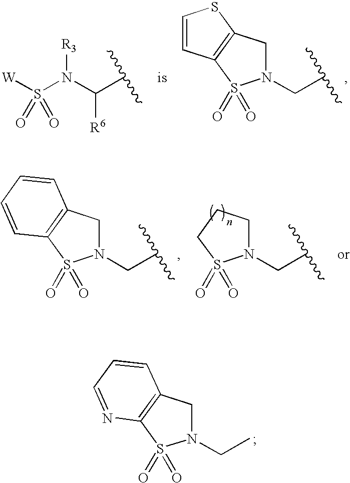
U is selected from the group consisting of O, NR³, S, and CR³₂; and
n is 0-5.

2.-12. (canceled)

13. The compound of claim 1, wherein, the moiety:

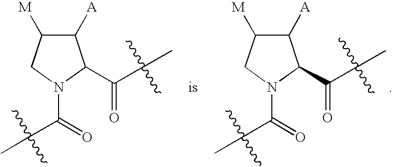

14. The compound of claim 1, wherein, the moiety:

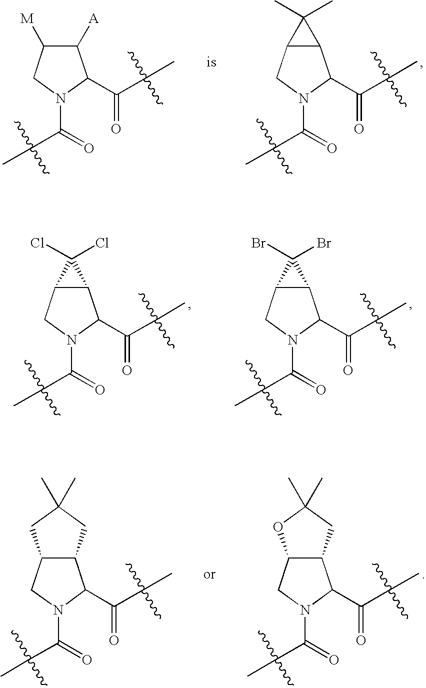

15. The compound of claim 1, wherein, the moiety:

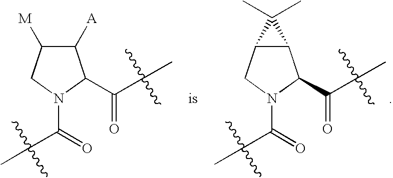

16.-19. (canceled)

20. The compound of claim 1, wherein, X is selected from the group consisting of 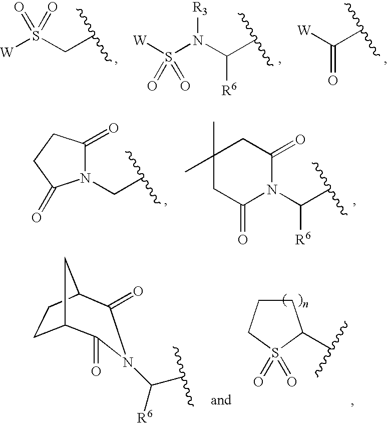
wherein W and R³, which can be the same or different, are independently alkyl or heteroaryl.

21. The compound of claim 1, wherein, X is 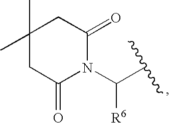
wherein R⁶ are two hydrogens.

22. The compound of claim 1, wherein, X is 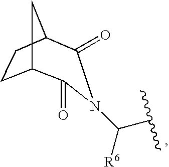
wherein R⁶ are two hydrogens.

23. The compound of claim 1, wherein, X is 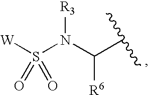
wherein W and R³ are each independently methyl and R⁶ are two hydrogens.

24. The compound of claim 1, wherein, X is 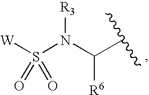
wherein W is pyridyl and R³ is methyl and R⁶ are two hydrogens.

25. The compound of claim 1, wherein, X is 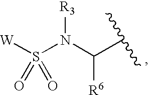
wherein W is 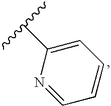
R³ is methyl and R⁶ are two hydrogens.

26. The compound of claim 1, wherein, X is 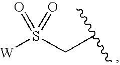
wherein W is tertiary butyl.

27. The compound of claim 1, wherein, U is NH.

28. A compound of the formula: 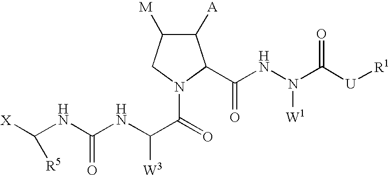
wherein the variable moieties are independently selected, further wherein R¹ is 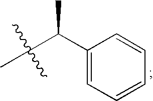
W¹ is propyl or cyclopropylmethyl;
the moiety: 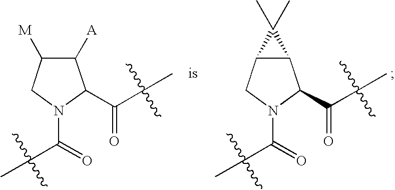
W³ is tertiary butyl, cyclohexyl, or 1-methylcyclohexyl;
R⁴ is hydrogen and R⁵ is tertiary butyl; or R⁴ and R⁵ together with the carbon to which they are attached form cyclohexyl;
X is 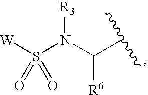
wherein W is methyl or 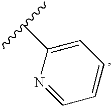
R³ is methyl, and R⁶ are two hydrogens or
X is 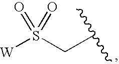
wherein W is tertiary butyl, 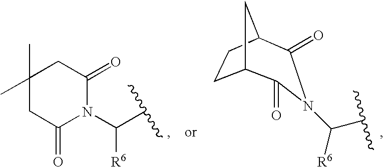
wherein R⁶ are two hydrogens;
and U is NH.

29. A pharmaceutical composition comprising as an active ingredient at least one compound of claim 1.

30. The pharmaceutical composition of claim 29 for use in treating disorders associated with HCV.

31. The pharmaceutical composition of claim 29 additionally comprising at least one pharmaceutically acceptable carrier.

32. The pharmaceutical composition of claim 31, additionally containing at least one antiviral agent.

33. The pharmaceutical composition of claim 32, still additionally containing at least one interferon.

34. The pharmaceutical composition of claim 33, wherein said at least one antiviral agent is ribavirin and said at least one interferon is α-interferon or pegylated interferon.

35. The pharmaceutical composition of claim 34, wherein said pegylated interferon is the PEG-Intron™ brand pegylated interferon.

36. A method of treating disorders associated with the HCV, said method comprising administering to a patient in need of such treatment a pharmaceutical composition which comprises therapeutically effective amounts of at least one compound of claim 1.

37. The method of claim 36, wherein said administration is oral or subcutaneous.

38. The use of a compound of claim 1 for the manufacture of a medicament to treat disorders associated with the HCV.

39. A method of preparing a pharmaceutical composition for treating the disorders associated with the HCV, said method comprising bringing into intimate physical contact at least one compound of claim 1 and at least one pharmaceutically acceptable carrier.

40. A compound exhibiting HCV protease inhibitory activity, or enantiomers, stereoisomers, rotamers, tautomers, and racemates of said compound, or a pharmaceutically acceptable salt, solvate or ester of said compound, said compound being selected from the compounds of structures listed below:

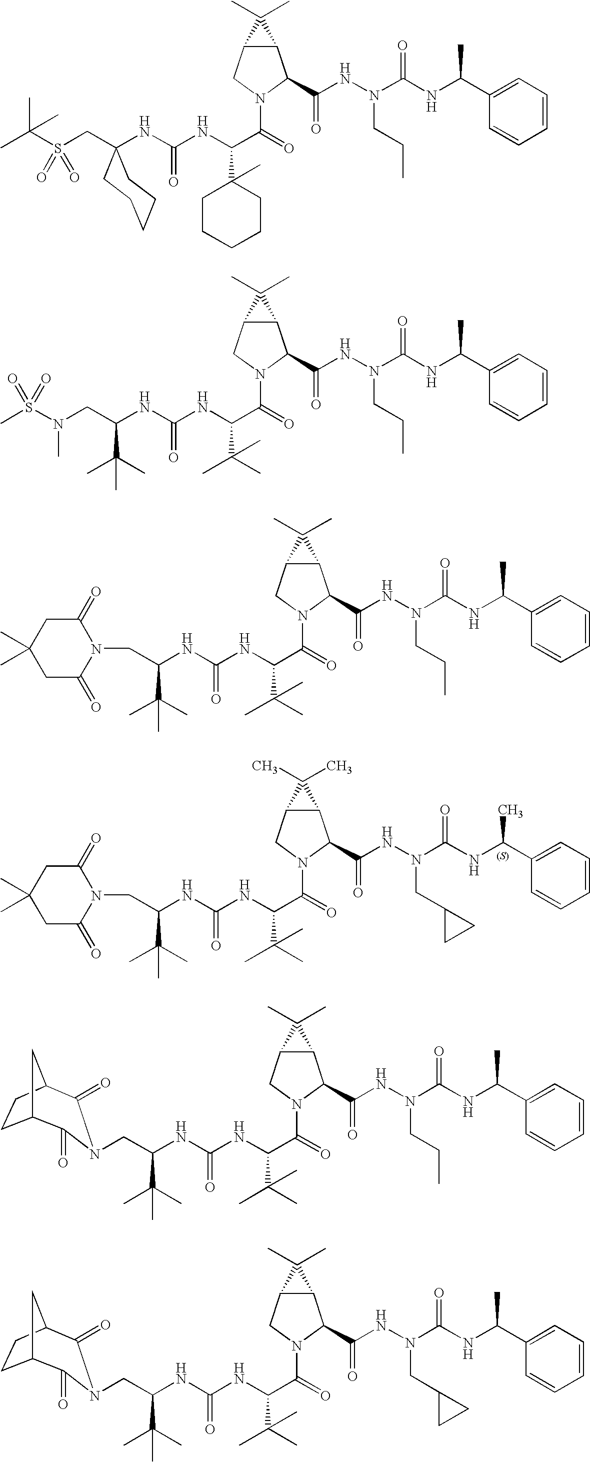

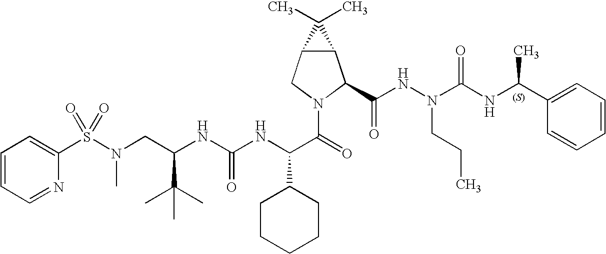

41. A compound of claim 1 in purified form.
